(19) United States
(12) Patent Application Publication
(10) Pub. No.: US 2010/0222289 A1
(43) Pub. Date: Sep. 2, 2010

(54) METHODS FOR DIAGNOSIS AND TREATMENT OF CHRONIC FATIGUE SYNDROME

(75) Inventor: Albert Martin Lerner, Birmingham, MI (US)

Correspondence Address: VENABLE LLP, P.O. BOX 34385, WASHINGTON, DC, 20043-9998, US

(73) Assignee: CFS Research LLC, Beverly Hills, MI (US)

(21) Appl. No.: 12/682,926

(22) PCT Filed: Oct. 22, 2008
(86) PCT No.: PCT/US2008/011995

§ 371 Date: Apr. 14, 2010

Related U.S. Application Data
(60) Provisional application No. 60/960,958, filed on Oct. 22, 2007.
(60) Provisional application No. 61/006,905, filed on Feb. 5, 2008.
(60) Provisional application No. 61/106,434, filed on Oct. 17, 2008.

(51) Int. Cl. A61K 31/7048; C12Q 1/70; A61K 31/52; C12Q 1/68; A61K 31/522; A61K 31/66; A61K 31/545; A61K 31/43; A61K 31/65; A61P 31/12

(52) U.S. Cl. 514/29; 435/5; 514/263.4; 435/6; 514/263.38; 514/120; 514/206; 514/197; 514/192; 514/154

(57) ABSTRACT

Methods for diagnosing and treating chronic fatigue syndrome in a patient comprise, for example, testing tissue of the patient for the presence of nucleic acid molecules of one or more CFS-causing herpesviruses and one or more non-herpesvirus infectious agents, and diagnosing the patient's CFS as (a) caused by one or more herpesvirus and no non-herpesvirus infectious agent; (b) caused by one or more herpesviruses and at least one non-herpesvirus infectious agent; (c) not caused by a herpesvirus and caused by at least one non-herpesvirus infectious agent; and (d) not caused by a herpesvirus and not caused by at least one non-herpesvirus infectious agent. In some embodiments, the methods of treating comprise administering a therapeutically effective amount of at least one pharmaceutical composition for each herpesvirus and/or non-herpesvirus infectious agent present found in the patient.

### FIELD OF THE INVENTION

The invention relates to methods for diagnosing and treating patients with chronic fatigue syndrome (CFS).

### BACKGROUND OF THE INVENTION

The chronic fatigue syndrome (CFS) is a public health problem with progressive invalidism in healthy young adults. CFS manifestations are life-altering fatigue in ordinary activities of daily living, including constellations of syncope, chest pain, muscle aches, palpitations, sore throat, low-grade fevers, inability to exercise without a worsening of all symptoms extending to the following day, cervical lymphadenopathy, cognitive impairment and resultant depression and an intolerance to alcohol. There is accompanying intense chronic immune activation. Severity of CFS is now evaluated based on the severity of fatigue, which is difficult to define, subjective and a prominent complaint in many disorders.

Spontaneous recovery rate for CFS patients is low, for example 19%. Cause and treatment have been elusive. Numerous treatment regimens have been attempted for CFS. Such attempted regimens have included administration of various agents such as immune stimulators and steroids, as well as recommending exercise and psychiatric treatment. Such treatment regimens are generally ineffective, though they may lead to modest improvement in symptoms. The various causes of CFS and how to distinguish them have not previously been known, and thus both observational and evidence-based trials have been misdirected or inappropriately planned.

Further information pertaining to administration of antiviral agents can be found in U.S. Pat. Nos. 5,872,123, 6,258,818, 6,399,622, 6,537,997, and 6,894,056, which are incorporated by reference in their entirety. These disclose treating chronic fatigue syndrome by administering one or more antiviral agents including, for example, valacyclovir, famcyclovir, cidofovir, ganciclovir, valganciclovir, and pharmaceutically acceptable derivatives and mixtures thereof, for example valacyclovir hydrochloride.

The prior patents describe diagnosing chronic fatigue syndrome by confirming the presence of a persistent cardiac viral infection, by evaluating the patient for the presence of particular antibodies to Epstein-Barr virus (EBV) and human cytomegalovirus (HCMV), and monitoring the patient for T-wave abnormalities through, for example, electrocardiographic monitoring or Holter monitoring.

Some CFS patients are treatable and reversible using single antiviral agents delivered in pharmacokinetic amounts for periods of about six months. But other patients are non-responsive. There is a pressing need for effective diagnostic techniques to determine the proper course of treatment for individual CFS patients.

### SUMMARY OF THE INVENTION

The embodiments of the invention can be used in the successful diagnosis and treatment of CFS patients, a major world-wide public health problem affecting over one million US citizens. The methods and other embodiments of the invention provide a method for subclassifying CFS patients, thus permitting the administration of appropriate therapies as determined by the subclass of CFS to which the patient belongs, leading to their recovery.

Serum IgM antibody to complete EBV, HCMV, and human herpesvirus 6 (HHV6) virions is usually absent in CFS, and the presence of positive serum IgG antibody to these viruses is interpreted by conventionally accepted standards as indicative of past, inactive, recovered infection. Therefore, EBV, HCMV and HHV6 have been considered to not be causally related to CFS, and adequate diagnostic techniques have been unavailable. Abortive herpesvirus infection, i.e., incomplete virus multiplication and/or assembly, has not been previously used as a target for diagnosing and treating CFS patients, for example with respect to HHV6 infection. According to embodiments of the invention, abortive herpesvirus infection is identified and treated in CFS patients.

Some patients suffering from CFS test positive for EBV, HCMV and/or HHV6 infection, either singly or in combination. Diagnosis and recognition of CFS has been accomplished using international and CDC criteria, presence of positive 24-Hour ECG (Holter) monitoring, an Energy Index point score less than 5, and conventional complete virus serologic identification of the involved herpesvirus. In some patients, serologic recognition of positive viral capsid antigen (VCA) IgM antibody titers for EBV infection, and the similar serologic recognition of positive IgM for HCMV p52 (UL44) and CM₂ (UL44 and UL57), has been possible. However, specific identification of active, abortive herpesvirus infection has not been possible.

In one aspect, the invention provides methods of diagnosing a patient with CFS. In some embodiments, the methods comprise the steps of determining the presence in the patient of primary abortive infection by one or more types of herpesvirus; and determining the presence in the patient of co-infection by one or more secondary non-viral infectious agents. The herpesvirus can be, for example, EBV, HCMV, HHV6, or combinations thereof. The secondary infectious agent can be, for example, Borrelia burgdorferi, Streptococcus pyogenes, Babesia microti, Mycoplasma pneumoniae, Ehrlichia chaffeensis or combinations thereof. Determining the presence of a secondary infectious agent can be done by any means known in the art, including, for example, those detailed herein.

Determining the presence of primary infection can comprise assaying for nucleic acid molecules that indicate the abortive replication of one or more CFS-causing herpesviruses, for example, assaying for expression of one or more middle (E or L) herpesvirus genes in a tissue of the patient. Assaying for expression of herpesvirus genes can be done by, for example, detecting mRNA of the gene, and/or by conducting an immunoassay that detects the protein products of the gene or antibodies to the protein products of the gene. This can be done at a time when immunological evidence of the CFS-causing agents cannot be detected. The nucleic acid molecules can be, for example, mRNA molecules. In some embodiments, the methods of the present invention can enable differentiating between HCMV infection and HHV6 infection. Determining the presence of secondary co-infection can be accomplished by, for example, assaying for nucleic acid molecules of one or more secondary infectious agents. In some embodiments, the methods can further comprise conducting Holter monitoring of the patient.

In some embodiments, the methods of the present invention can comprise determining if the patient meets the criteria for CFS as established by the International Chronic Fatigue Syndrome Study Group and determining if the patient has not exhibited any significant improvement in the previous six months. The methods of the present invention can be repeated after, for example, at least about 2 weeks.

In another aspect, the invention provides methods of diagnosing CFS in a patient, comprising selecting a set of target serologic markers of a plurality of pathogens, wherein the pathogens are associated with CFS; obtaining a set of quantitative values for a reference level for each of the serologic markers in the set, wherein a level of serologic marker above the reference level indicates the presence of a pathologic level of the pathogen in the physiological fluid tested; obtaining from the patient a sample of physiological fluid in which the target serologic markers would be found if the pathogen is present in the patient; measuring the serologic levels for each of the target serologic markers in the physiological fluid of the patient and obtaining a quantitative value for the serologic level of each target serologic marker; comparing the serologic level with the reference level for each target serologic marker; and identifying the patient as having CFS if the serologic level is significantly above the reference level. The pathogen can be one or more of, for example, HCMV, EBV, HHV6, Borrelia burgdorferi, Streptococcus pyogenes, Babesia microti, Ehrlichia chaffeensis, Mycoplasma pneumoniae, and/or combinations thereof.

In still another aspect, the invention provides methods of treating a CFS patient who is diagnosed according to methods outlined herein. In some embodiments, the methods comprise administering to the patient an amount of at least one antiviral agent effective to treat each primary infection found in the patient, and administering to the patient an amount of at least one pharmaceutical composition effective to treat each co-infection found in the patient, for a period of time effective to treat the CFS. The antiviral agent can be one or more of, for example, valacyclovir, valganciclovir, maribavir, foscarnet, and/or combinations thereof. The pharmaceutical composition can comprise an active agent such as, for example, ceftriaxone, amoxicillin, penicillin G, ataquavone, doxycycline, azithromycin and/or combinations thereof. In some embodiments, the methods further comprise monitoring the patient over a time course of treatment. This monitoring can comprise, for example, observing the patient for side effects resulting from the administration of the at least one antiviral agent; and/or determining the level of primary abortive infection by one or more types of herpesvirus after treatment, and adjusting the dosage level of the at least one antiviral agent accordingly. In some embodiments, the monitoring can comprise, for example, conducting an immunoassay on a tissue sample of the patient to determine the presence or absence of antibodies to gene products of CFS-causing herpesvirus and/or non-viral infectious agents. The gene product can be, for example, an mRNA or protein produced during abortive replication of the one or more types of herpesvirus. In some embodiments, the methods can be repeated, for example at least every 6 months, until the patient lacks symptoms of CFS.

In still another aspect, the invention provides methods for identifying a CFS patient who will respond to antiviral therapy. In some embodiments, the methods comprise the steps of obtaining from the patient a sample of a body fluid; extracting nucleic acid molecules from the sample; amplifying the nucleic acid molecules; detecting target nucleic acid molecules or portions thereof that indicate the presence of one or more CFS-causing herpesviruses; and identifying an antiviral therapy for the patient. Detecting nucleic acid molecules or portions thereof can comprise identifying expression of one or more middle (E or L) herpesvirus genes, and/or detecting mRNA of the gene. The one or more CFS-causing herpesviruses can be, for example, HCMV, EBV, HHV6, or combinations thereof. The body fluid can be, or example, blood. In some embodiments, amplifying the nucleic acid is done using reverse transcriptase polymerase chain reaction (RT-PCR). Detecting nucleic acid sequences can be done using, for example, a target-specific primer extension (TSPE). The methods can further comprise confirming the presence of nucleic acid sequences using confirmatory RT-PCR. These methods can provide a sensitivity of detecting herpesvirus nucleic acid molecules that is higher than the sensitivity of immunological assays for the herpesvirus.

In some embodiments, the methods can comprise analyzing the blood for the presence of IgM serum antibodies to the gene products, for example by conducting an assay for antibodies to HCMV gene products CM₂ and p52, by using an assay, such as, for example, the Copalis Immunoassay.

In still another aspect, the invention provides methods of determining a therapeutic regimen for chronic fatigue syndrome in a patient, comprising testing tissue of the patient for the presence of nucleic acid molecules of one or more CFS-causing herpesviruses and one or more non-viral infectious agents, and categorizing the patient's CFS as (a) caused by one or more herpesvirus and no non-viral infectious agent; (b) caused by one or more herpesviruses and at least one non-viral infectious agent; (c) not caused by a herpesvirus and caused by at least one non-viral infectious agent; and (d) not caused by a herpesvirus and not caused by at least one non-viral infectious agent. The nucleic acid molecules can be, for example, mRNA and can comprise gene products from early or middle genes that are expressed during abortive herpesvirus infection. The CFS-causing herpesvirus can be, for example, one or more of HCMV, EBV, and/or HHV6. In some embodiments, the methods can comprise testing for the presence of one or more mRNA molecules that correspond to genes such as, for example, EBV BMRFI, EBV BL-L3, EBV EC-R1, EBV EC-L1, HCMV UL44 polymerase processivity factor, HCMV UL57 major DNA binding protein, HCMV UL83 virion transactivator, HCMV UL97 phosphotransferase, HHV6 UL27, HHV6 U41, HHV6 U54, HHV6 UL69, and/or combinations thereof. The methods can comprise administering at least one antiviral agent such that each herpesvirus found in the patient is effectively treated by at least one antiviral agent administered to the patient. The methods can also comprise administering at least one pharmaceutical composition such that each non-herpesvirus infectious agent found in the patient is effectively treated by at least one pharmaceutical composition administered to the patient.

In come embodiments, the methods comprise monitoring the patient over a time course of treatment. This monitoring can comprise, for example, assaying for a decrease in the patient's levels of viral gene products, and adjusting the dosage level of the at least one antiviral agent and/or the at least one pharmaceutical agent accordingly. The time course of treatment may be at least or about 6 or nine months, at least or about one, one and a half, two, three years or longer, until the patient evidences demonstrable improvement based on medical examination, EIPS scores, serological evidence, or otherwise as may be determined by a practitioner following the methods of the invention.

In yet another aspect, the invention provides kits. The kit can comprise, for example, means for detecting serologic evidence of HCMV; means for detecting serologic evidence of EBV; means for detecting serologic evidence of HHV6; and means for detecting serologic evidence of at least one non-viral pathogen selected from the group consisting of Borrelia burgdorferi, Streptococcus pyogenes, Ehrlichia chaffeensis, Babesia microti, and Mycoplasma pneumoniae.

### BRIEF DESCRIPTION OF THE DRAWINGS

FIG. 1 is a diagram demonstrating that TSPE primers contain both a virus-specific oligonucleotide sequence and a tag oligonucleotide that hybridizes to a complementary anti-tag oligonucleotide bound to a spectrofluorometrically labeled microsphere.

FIG. 2 presents a comparison of Energy Index Point Score (EIPS) for those that responded to treatment (i.e., “responders”) versus all CFS patients in a study at three-month intervals. FIG. 2A is a graph depicting change in EIPS over time for 90 “responders.” FIG. 2B is a graph depicting change in EIPS over time for all CFS patients in the study (n=124).

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference as if each had been individually incorporated.

“Diagnosing” encompasses, for example, identifying a CFS patient who will respond to treatment with an antiviral and/or a non-antiviral therapy.

“Determining the presence of an infection” in a patient also encompasses determining the absence of the infection. In other words, “determining the presence of an infection” encompasses method for assaying or evaluating evidence of the presence or absence of an infection in a patient by various analytical techniques. Similarly, “detecting the presence” of, for example, a pathogen or physiological marker also encompasses detecting its absence. As used herein “testing” for the presence of a pathogen or physiological marker encompasses determining the presence or absence of the pathogen or physiological marker. Such testing can be done using any means known in the art.

Assaying for nucleic acid molecules includes probing, amplifying, and other techniques known to persons of ordinary skill for determining the presence or absence of particular nucleic acids, e.g. those found in genes of an infectious agent, in tissue of a patient, e.g. serological samples.

As used herein, “treatment” refers to the prevention, partial alleviation or cure of the condition or disorder, or at least one symptom of the condition or disorder. As used herein, “effective” or “therapeutically effective” means sufficient to cause at least one of a patient's symptoms to decrease in frequency and/or intensity. The course of recovery of CFS patients under treatment can be monitored by observing an increase in the Energy Index point score (EIPS) of 1.0 or more units, and/or a decrease in serological indices of pathogens.

As used herein, “primary abortive infection” includes, for example, first episode infection with EBV, HCMV and HHV6. As used herein, “primary abortive infection” and “primary infection” are substantially equivalent terms. As used herein, “co-infection” includes infection with, for example, Borrelia burgdorferi, Streptococcus pyogenes, Ehrlichia chaffeensis, Babesia microti and Mycoplasma pneumoniae. “Co-infecting agent” means, for example, Borrelia burgdorferi, Streptococcus pyogenes, Ehrlichia chaffeensis, Babesia microti and Mycoplasma pneumoniae. As used herein, “infection” means the invasion of a host organism's body by another organism or entity, for example a virus or bacteria. Infection by a virus may, but does not necessarily, include entry of the virus into host cells, production of gene products based on the viral nucleic acid, replication of the virus, and/or further spread of the virus within the host body, which may or may not induce an immunological response by the host organism. “Infection” may include the latent presence of virus, for example that which is not replicating, and whose genes are not being expressed; or, more typically, infection may include a virus at least some of whose genes are being transcribed into mRNA, which may be translated into protein gene products.

“Infection” includes abortive infection and/or replication. As used herein, “abortive” refers to infections characterized by incomplete viral replication, for example with nonassembly into a complete virion. Abortive infection can include, for example, expression of the virus genome to produce early (IE), middle (E) or late (L) gene products including, for example, EBV viral capsid antigens. In such an example of abortive infection, the gene products are not assembled into a complete virus. Abortive infection may include, primarily or exclusively, early only, early and middle, or early, middle and late gene products. Abortive herpesvirus infection is a pathogenic mechanism of CFS that can be used to diagnose the disease, and to identify patients who are good candidates for antiviral therapy. As used herein, “primary abortive infection” includes, for example, infection with EBV, HCMV and HHV6. As used herein, “secondary nonviral infectious agent” includes, for example, Borrelia burgdorferi, Streptococcus pyogenes, Ehrlichia chaffeensis, Babesia microti and Mycoplasma pneumoniae. Streptococcus pyogenes infection manifests itself as Adult Rheumatic Fever. As used herein, “secondary infectious agent” and “secondary nonviral infectious agent” are substantially equivalent terms.

As used herein, “antiviral agent” includes, for example, valacyclovir, valganciclovir, maribavir, famciclovir and foscarnet. However, any antiviral agent that is effective against a CFS-inducing infection can be used according to the methods disclosed herein.

As used herein, “amplify” means to increase the number of molecules, for example nucleic acid molecules, in a sample. For example, polymerase chain reaction (PCR) or reverse transcriptase polymerase chain reaction (RT-PCR) methods can be used to amplify nucleic acid molecules in a sample. As used herein, “amplicon” means a piece of nucleic acid, for example DNA, that has been produced using amplification techniques, for example PCR or RT-PCR. As used herein, “immunoassay” includes, for example, enzyme-linked immunosorbent assay (ELISA).

As used herein, “phosphotransferase” is meant to be substantially equivalent to “kinase.” As used herein, “gene products” includes, for example, mRNA and protein molecules that emanate from a locus in an organism's genome.

The amount of antiviral agent required to constitute a therapeutically effective amount will vary based on a number of factors, including the severity of the chronic fatigue syndrome; the identity, age, body weight, general health, gender, diet and chemical make-up of the patient; the type and degree of the cellular response to be achieved; the specific agents or composition employed, and its activity; the time of administration, route of administration, and rate of excretion of the agent; the duration of the treatment; drugs used in combination or coincidental with the specific agent; and like factors well known in the medical arts. It must also take into consideration the therapeutic window, that is, the need to adjust and minimize toxic side effects. For example, it is well within the skill of the art to start doses of the agents at levels lower than those required to achieve the desired therapeutic effect and to gradually increase the dosages until the desired effect is achieved.

The chronic fatigue syndrome (CFS) was recognized as a public health problem because of recurrent signs and symptoms of this previously unknown life-altering illness. Young adult women (4 to 1 man) are affected. Although efforts to understand cause and allow etiologic-based treatment continue, no cause or established treatment has been found. Cardiac, immune dysfunction, and radiographic cerebral abnormalities have been identified. The average death age of 144 CFS registered by the National CFIDS Foundation was 39.3 years and 20.1% died of suicide and 20.1% of heart failure. The paradigm for the inventive methods is that the chronic fatigue syndrome (CFS) is caused in some patients by Epstein-Barr virus (EBV), human cytomegalovirus (HCMV) and Human Herpesvirus 6 (HHV6) in single or multiple virus infection. This paradigm affirms that the Herpesviruses, despite maximum efforts from these immunocompetent affected patients, continue an incomplete abortive multiplication of middle gene products, usually without achieving complete virus synthesis. Thus, the invention involves treating herpesvirus CFS subsets with long-term antiviral use of valacyclovir for EBV subsets and valganciclovir for HCMV or HHV6 subsets. These herpesvirus subsets, we designate Group A CFS.

CFS is generally defined as a disorder of uncertain cause that is characterized by persistent profound fatigue usually accompanied by impairment in short-term memory or concentration, sore throat, tender lymph nodes, muscle or joint pain, and headache unrelated to any preexisting medical condition and that typically has an onset at about 30 years of age. MedlinePlus Medical Dictionary, http://www.nlm.nih.gov/medlineplus/mplusdictionary.html (last visited Feb. 1, 2008). As used herein, CFS is defined to be a disorder caused by infection with a CFS-causing agent. “CFS-causing agent” includes CFS-inducing herpesviruses, for example, HCMV, EBV, and/or HHV6. “CFS-causing herpesvirus” includes, for example, HCMV, EBV, and/or HHV6. CFS is often characterized by abortive replication and an inability on the part of the patient to inactivate the CFS-causing agent by inducing, for instance, inactive herpesvirus latency. CFS patients can respond to antiviral therapy, as measured by, for example, a reduction in nucleic acid gene products of one or more CFS-causing agents. It may be necessary to treat a co-infection in order for the effectiveness of antiviral therapy to be apparent.

According to some embodiments of the invention, it has been determined that CFS is caused by a persistent abortive herpesvirus infection caused by EBV, HCMV and/or HHV6, either singly or in combination. These infections often involve the heart and generally can best be treated with a specifically designed antiviral treatment regimen that takes into account which viruses are present and targeting each one with appropriate antiviral agents. Furthermore, it has surprisingly been discovered that CFS can also involve co-infection with, for example, Borrelia burgdorferi, Streptococcus pyogenes, Ehrlichia chaffeensis, Babesia microti and Mycoplasma pneumoniae. Each infection found to be present in a patient must be treated or the therapeutic regimen will be unsuccessful. “Determining a therapeutic regimen” encompasses prescribing and/or administering one or more pharmaceutical compositions containing one or more active agents such that each infection that is present in the patient and contributing to the etiology of CFS is treated. Several years of treatment may be necessary. When the appropriate treatment regimen is applied, CFS patients recover their health.

Since serum antibodies to nonstructural gene products (for example, the tegument) of middle, late or, at times, early herpesvirus genes are not present in the blood of patients after known infection with herpesviruses (for example EBV, HCMV and/or HHV6), one would not expect serum antibodies to these early, middle or late gene products in CFS patients. Furthermore, the presence of IgM serum antibody titers to early, middle or late gene products of EBV, HCMV, or HHV6, not present after usual recovery from infections by the aforementioned herpesviruses, is diagnostic of CFS.

It has surprisingly been found that middle gene products of the herpesviruses HCMV, HHV6 and/or EBV, for example those from genes later than gene 30 of the viral genome, or for example the products of from about gene 50 to about gene 125, or for example genes up to or at least about 60, 70, 80, 90, 100, 110, or 120, are found in patients with CFS and are absent in patients who do not have CFS. Middle gene products are those that are at least partially expressed in patients that exhibit symptoms of CFS but not in those who do not exhibit symptoms of CFS. These genes can be described as middle gene products or middle to mid-late gene products. The gene sequences of the herpesviruses HCMV, HHV6 and EBV are known. Their genomes have been mapped. A person of skill in the art would understand which genes of HCMV, HHV6 and EBV are described herein. As used herein, “about” may refer to a range from 10% below the referenced number to 10% above the referenced number. For example, “about 50” may mean from 45 to 55. Other meanings of “about” may be apparent from the context.

The fact that middle gene products of the herpesviruses HCMV, HHV6 and/or EBV are found in patients with CFS and are absent in patients who do not do not have CFS has never previously been known or suggested. Abortive infection can involve interruption of the viral replication process at any point in the process. In CFS patients, viral replication is interrupted during expression of middle or middle to late genes. Many people have antibodies to herpesvirus gene products present in their body fluids or tissues. For example, gene expression of early gene products occurs in people without CFS. However, middle gene products, for example HCMV gene products p52 and CM₂, are generally found in CFS patients. Expression of middle, and middle and late, genes, but without completion of viral replication, is associated with CFS and is treatable with appropriate antiviral agents effective against the infecting viruses. Patients without such infection can be distinguished for other treatment, as can patients with other infections that may contribute to CFS symptoms.

The invention provides methods for treating CFS by sub-classifying CFS patients into two groups. Group A includes patients with persistent abortive infection with Epstein-Barr virus (EBV), human cytomegalovirus (HCMV) and/or human herpesvirus 6 (HHV6) with no additional co-infections. Group B includes patients with persistent abortive EBV, HCMV and/or HHV6 infection with co-infections by another agent. Such co-infections include, for example, infection by Borrelia burgdorferi, Babesia microti, Ehrlichia chaffeensis, Streptococcus pyogenes and/or Mycoplasma pneumoniae. These two groups of CFS patients meet the present CDC diagnosis criteria. See U.S. Prov. Appl. No. 60/960,958, entitled “Method of Treating Chronic Fatigue Syndrome in Co-infection,” and U.S. Prov. Appl. No. 61/006,905, entitled “Methods for Diagnosis of Chronic Fatigue Syndrome Using Viral Gene Products,” which are incorporated by reference in their entirety and for all purposes. The invention also contemplates the use of any of the assays, antiviral agents or pharmaceutical compositions

Herpesvirus involvement in CFS etiology is evidenced by the fact that CFS patients recover after long-term administration of specific pharmacokinetic-directed antiviral therapy. This remarkable “single-element reversibility” provides evidence of herpesvirus involvement in CFS etiology, as shown by the following: in Group A CFS patients with EBV single virus infection, administration of valacyclovir (Valtrex) leads to recovery. Human EBV causation of CFS is also supported by the fact that valacyclovir is not an effective therapeutic antiviral agent versus HCMV or HHV6. The minimum inhibitor concentration (MIC) of valacyclovir to EBV is 1.5-3.0 μg/ml.

Additional evidence for the viral involvement in the etiology of CFS is provided by the following findings: elevated mean values of bioactive 2-5A RNase L activity in peripheral blood mononuclear cells; brain abnormalities at MRI; increased immune reactivity involving heightened T-cells and natural killer cells consistent with a virus etiology; abnormal tilt-table studies, 24-Hr. Holter monitorings, and cardiomyopathic findings.

Furthermore, in Group A CFS patients where CFS is caused by HCMV and/or HHV6, it is difficult to distinguish the causative herpesvirus because valganciclovir (valcyte) is effective versus both HCMV and HHV6 viruses. The MIC of valganciclovir to HCMV and HHV6 is 0.15-0.3 μg/ml.

Previously, there was no method, with the exception of detecting serum IgM antibody to EBV VCA or HCMV p52 or CM₂, to determine definitive herpesvirus active infection prior to antiviral treatment. Accordingly, the cause of a patient's CFS could not be determined until and unless these serologic markers appeared in the patient's blood. Furthermore, there was no straightforward method to definitively determine which one or more of the three CFS causing agents were actually causing CFS in a given patient. Here, we provide methods for definitive determination of EBV and/or HCMV and/or HHV6 infection in CFS patients.

Given the incomplete and inconsistent scientific views about the etiology, diagnosis and treatment of CFS as a viral infection, it could not previously have been predicted that gene expression analysis for herpesvirus gene products could provide a definitive diagnosis and basis for prescribing an effective therapeutic regimen for CFS patients. In particular, prior knowledge relating to immunological assays was not predictive of success with the methods of the invention because the pathways of herpesvirus gene expression in infected humans are highly complex and unpredictable. Previously known techniques would not provide the advantages of early and definitive detection of treatable viral infection as causative of CFS.

The chronic fatigue syndrome is a major public health problem which has had no established cause, and no established treatment. The complexity of three herpesviruses multiplying, usually abortively in single and multiple infections often with confounding masquerading co-infections with tick-borne infections of Lyme disease, Babesiosis and Ehrlichiosis, or, in turn, Adult Rheumatic Fever (caused by Streptoccocus pyogenes) or chronic Mycoplasma pneumoniae myocarditis has challenged placebo-controlled evidence-based trials of therapy. The herpesvirus CFS accompanied with Lyme disease, Babesiosis, Ehrlichiosis, Adult Rheumatic Fever and/or Mycoplasma pneumoniae myocarditis comprise Group B CFS disease. Experience indicates that six to eight months of antiviral therapy may be necessary before the often remarkably beneficial result may be experienced. The specific pharmacokinetic administration of valacyclovir, famciclovir and/or valganciclovir is strikingly therapeutic. With antiviral treatment, the Energy Index Point Score (EIPS or EI) increases, and symptoms of CFS lessen and in many cases disappear.

The Energy Index point score (copyright, Lerner A M and Deeter R G, 1999), is a simple and reliable metric that can be used to easily evaluate the functional capacity of the CFS patient at each patient-physician visit. The EI is measured on a scale from 1 to 10. Validation of the EI was done using two methods: a) 20 CFS patients and 22 healthy adults, matched for sex, age, place and time; EI, CFS=3.6; EI, healthy adults=9.9, p=<0.0001, and b) 55 CFS patients evaluated at the same time by the EI and Fatigue Severity Score, correlation 0.67, p=0.0066. Improvement to disappearance of CFS symptoms correlates with an increasing EI.

The validated Energy Index point score generally can be calculated for each CFS patient every 3 months at physician visits. A CFS patient has an EI≦5. A CFS patient with an EI of 0 is bedridden; a CFS diagnosis is no longer present at an EI>5. The EI effect size is 0.25, a medium effect size is 0.5. A large effect size is >0.8.

TABLE 1
CFS Energy Index Point Score*.

| | Energy Expenditure | |
|---|---|---|
| Grade | (Kcal per day**) | Activity |
| 0 | 1715 | Bedridden, up to bathroom |
| 1 | 1750 | Out of bed, 30 min to 1 h/day |
| 2 | 1785 | Out of bed, 1-2 h/day |
| 3 | 1855 | Out of bed, 2-4 h/day |
| 4 | 1925 | Out of bed, 4-6 h/day |
| 5 | 1995 | Can work at sedentary job, 40 h/week |
| | | with difficulty exhausted at completion |
| | | of working day |

Recovery

| | | |
|---|---|---|
| 6 | 2083 | May maintain a 40-h sedentary work |
| | | week, plus light, limited housekeeping |
| | | and/or social activities. Daily naps |
| | | are necessary |
| 7 | 2205 | Up 7 a.m. to 9 p.m. Able to work a |
| | | sedentary job plus light housekeeping. |
| | | No naps necessary |
| 8 | 2240 | Able to manage full work (sedentary) |
| | | plus manage a household. No naps |
| | | necessary |
| 9 | 2450 | May exercise at approximately ½ |
| | | to ⅔ normal without excessive |
| | | fatigue |
| 10 | >2500 | Normal |

*The CFS Energy Index point score can also be determined by questionnaire (US copyright, 1999, Lerner A. M., Deeter, R. J.) Reproduced with permission
**Kcal per day is calculated for a 70 kg CFS patient.

Valacyclovir, famciclovir and valganciclovir can be safe and effective CFS medications. Valganciclovir-receiving patients can experience reversible increases in levels of aminotransferases. In such cases, valganciclovir administration can be ceased, and resumed at an altered dosage when aminotransferases return to normal levels. Concomitant medicines with hepatobiliary metabolism including non-steroidal anti-inflammatory medicines and cholesterol-lowering medicines are often stopped at onset of antiviral nucleoside administration. Valganciclovir can be administered at 450 mg once daily dose for three days before increasing to full dosage.

Many patients with Group A herpesvirus CFS benefit from valacyclovir/famciclovir and/or valganciclovir administration. Famciclovir can be substituted for valacyclovir at the identical dosage. CFS patients reaching an EIPS of greater than 7 live normal lives. At an EIPS of greater than 5, patients no longer meet criteria for the diagnosis of CFS (although they may have fatigue symptoms). Under the above-described pharmaceutical regimen, CFS patients can achieve EIPS of 7, 8 or 9. This is the case even where the patients had suffered from CFS for long periods of time, for example approximately 5 years, prior to the beginning of the therapeutic regimen. These data strongly support both the fact that CFS can be caused by herpesvirus infection, and that such CFS is reversible by long-term antiviral therapy.

It is not uncommon for elevated serum IgM antibody titers to structural viral antigens to HCMV or HHV6 to not appear in CFS patients, though some do exhibit elevated serum antibodies to EBV p-18 recombinant VCA. However, it is more common for CFS patients to exhibit elevated serum IgM antibody titers to non-structured HCMV tegument gene products p52 and CM₂, signifying abortive HCMV virus multiplication. Abortive herpesvirus infection, which encompasses, for example, a failure to achieve herpesvirus latency, would account for the apparent paradox of no structural elevated IgM serum antibody titers. Further, we and others have attempted to isolate complete virus; recognize viral antigens; and demonstrate positive PCR virus responses in bloods, urines, tissues or cardiac biopsies. The challenge of the pathogenicity of abortive herpesvirus multiplication is clearly raised by these results. CFS patients in large numbers are markedly benefited after appropriate classification to Group A, plus appropriate subset and then long-term antiviral administration.

Methods of Diagnosis

In some embodiments, the invention provides methods of diagnosing, treating and monitoring a patient with CFS during a time course of treatment until the patient reaches a satisfactory specific clinical endpoint. Monitoring the patient involves serological tracking of the decrease in the patient's levels of viral gene products and adjusting pharmacological treatment accordingly. It also involves observing the presence of side effects to the pharmacological treatments. Many antiviral agents that are effective against CFS have a small therapeutic window; i.e., the range of dosages that are efficacious and do not have unacceptable side effects or toxicities is relatively small. Monitoring the patient permits the clinician to closely tailor dosage levels of such antiviral agents to the patient's need for them while also avoiding or minimizing side effects or toxicities. Monitoring the patient permits the dosage level of, for example, an antiviral agent to be adjusted based on various factors. Monitoring the patient can also involve determining, or obtaining or causing to be determined, the level of primary abortive infection by one or more types of herpesvirus after treatment, and adjusting the dosage level of the at least one antiviral agent accordingly. As used herein, “adjusting dosage level accordingly” can encompass adjustment of the dosage level up or down. In some embodiments, the methods of diagnosis involve producing a list of results of the assays conducted in printed or other readable form.

According to some embodiments of the invention, the patient samples that are tested may be blood, lymph, or another body fluid, solid tissue, cells, or other biological material, all of which are considered to be patient tissue. The samples can be obtained via biopsy, for example of cardiac tissue.

The methods of diagnosing CFS in a patient can also involve obtaining from the patient a sample of a body fluid or other tissue in which serologic evidence of the presence of nucleic acid molecules that indicate infection by one or more CFS-causing agents would be detected; extracting nucleic acid from the sample; amplifying the nucleic acid; and detecting nucleic acid sequences, each of which specifically indicates the presence of a CFS-causing agent. The body fluid can be, for example, blood, for example the serum and circulating white blood cells in blood, though any body fluid or tissue that would provide evidence of the presence of nucleic acid molecules that indicate infection by one or more CFS-causing agents can be used. The body fluid can be obtained by any means known in the art. As used herein, “body fluid” and “physiological fluid” are substantially equivalent terms. Mononuclear phagocytes and B- and T-lymphocytes are known to harbor EBV, HCMV and HHV6 and to circulate in the blood. Amplifying the nucleic acid can be done using, for example, RT-PCR. Detecting nucleic acid sequences can be done using, for example, TSPE. Extraction can be done using any means known in the art. These methods can further comprise confirming the presence of herpesvirus nucleic acid sequences using confirmatory RT-PCR. An advantage of this aspect of the invention is that assaying for nucleic acid molecules can be conducted at a time when immunological evidence of the CFS-causing agents cannot be detected. The methods of diagnosis described herein can all be employed in conjunction with any method of treatment disclosed herein.

A further aspect of the invention relates to methods of diagnosing CFS in a patient, comprising evaluating the patient for serologic evidence of the presence of nucleic acid molecules that are indicative of infection by one or more CFS-causing agents. The nucleic acid molecules can be, for example, mRNA molecules, and the mRNA molecules can be, for example, those corresponding to the following genes/gene products. For EBV: EBV BMRFI, EBV BL-L3, EBV EC-R1, EBV EC-L1; for HCMV: HCMV UL44 polymerase processivity factor, HCMV UL57 major DNA binding protein, HCMV UL83 virion transactivator, HCMV UL97 phosphotransferase, HCMV UL97 ganciclovir kinase; and for HHV6: HHV6 UL27, HHV6 U41, HHV6 U54, HHV6 UL69, and combinations thereof. Assays for other early, middle or late gene products of EBV, HCMV or HHV6 are included in this invention, as the ordinary organized sequence of multiplication is interrupted by the host cell, typifying abortive infection in CFS patients. Abortive multiplication involves interruption of the orderly sequence of herpesvirus multiplication by the host cell and can occur at early, middle or late genes.

The method of messenger RNA detection allows direct monitoring of herpes viral gene expression in abortive multiplication from a latent state in which there is either 1) no viral gene expression (complete latency); or 2) only “immediate” early herpesvirus gene expression, which does not produce CFS disease. Data suggest up to or at least 30 early genes are processed to produce CFS. The fact that early herpesvirus gene expression does not produce CFS disease is demonstrated by data showing that equal numbers of patients with EBV CFS and healthy patients have elevated serum antibody titers to very early antigen EBV, Early Antigen (EA), Diffuse. In CFS patients with HCMV CFS, p52 and CM₂ (UL44 and UL57) elevated antibody titers indicate viral expression at least to mid-gene level among the approximately 200 genes in the three herpesviruses implicated in CFS.

The use of multiplex real time quantitative RT-PCR allows for recognition of the exact temporal multiplication of specific genes in circulating white blood cells of CFS patients. The presence of RNA transcripts to early and late herpesvirus genes is indicative of active abortive infection. The invention permits the simultaneous determination of the presence of messenger RNA to several early, middle and late genes from each of EBV, HHV6 and HCMV, using TacMan and Light [Cycler] from a single sample of blood, using separated leukocytes of CFS patients. The genes initially targeted by RT-PCR and ELISA assays according to this embodiment include, without limitation, those listed in Table 1. This invention relates to abortive herpesvirus infection involving any of the genes of EBV, HCMV or HHV6 in abortive incomplete multiplication. The genes can include, for example, single or fused gene products.

TABLE 2
Examples of Targeted Genes

| EBV (35) | HCMV | HHV6 |
|---|---|---|
| BMRFI | UL44 polymerase processivity factor | UL27 |
| BL-L3 | UL57 major DNA binding protein | U41 |
| EC-R1 | UL83 viriontrans activator | U54 |
| EC-L1 | UL97 phosphotransferase/kinase | UL69 |

According to some embodiments of the invention, other early, middle, and late gene products can be assessed. A person of ordinary skill may select the targeted gene and design a probe and method for determining its presence or absence in a tissue sample of a patient. Herpesvirus genes generally belong to one of two main categories: latent or lytic. Three generally kinetic classes of genes exist: immediate early (IE) genes that provide functions for regulation of gene expression: early (E), also referred to as middle here, genes that encode proteins needed for DNA replication; and late (L) genes that encode virion proteins. Middle and middle to late genes therefore encompass some E and some L genes.

Thus by this single amplified RT-PCR method, the identity of the one or more herpesviruses that are causing CFS can be elucidated definitively, which can be used to direct initiation of specific antiviral therapy. For example, (1) EBV CFS as demonstrated by RT-PCR would indicate therapy with, for example, valacyclovir; (2) HCMV and/or HHV6 CFS as determined by RT-PCR would indicate therapy with, for example, valganciclovir; and (3) HCMV and/or HHV6 plus EBV CFS would indicate treatment with, for example, both valganciclovir and valacyclovir. New antiviral drugs with different or similar modes of action can be substituted for those listed above.

In another aspect of the invention, gene products may be fused to give a broader array of gene product-serum antibody reactivity. For instance, for HCMV CFS, assaying for the gene product CM₂, which results from the fusion of UL44 and UL57, can be more sensitive than assaying for either UL44 or UL57 alone.

In another aspect, the invention provides methods of diagnosing CFS in a patient, comprising selecting a set of target serologic markers of a plurality of pathogens, wherein the pathogens are associated with CFS; obtaining a set of quantitative values for a reference level for each of the serologic markers in the set, wherein a level of serologic marker above the reference level indicates the presence of a pathologic level of the pathogen in the physiological fluid tested; obtaining from the patient a sample of physiological fluid in which the target serologic markers would be found if the pathogen is present in the patient; measuring the serologic levels for each of the target serologic markers in the physiological fluid of the patient and obtaining a quantitative value for the serologic level of each target serologic marker; comparing the serologic level with the reference level for each target serologic marker; and identifying the patient as having CFS if the serologic level is significantly above the reference level. As used herein, a serologic level is significantly above the reference level if the serologic level exceeds the reference level to the degree that it would indicate the presence of an infection to a person of ordinary skill in the art. “Serologic marker” encompasses any serologic evidence that indicates the presence of the pathogen in the patient's body. Such evidence can include, for example, the presence of a molecule or other entity that is generally not present in a healthy individual; an increase or decrease in the level of a molecule or other entity over what is generally present in a healthy individual; or any other indicator known in the art. “Pathogens” encompasses those associated with the etiology of CFS, including, for example, one or more of HCMV, HHV6, EBV, Borrelia burgdorferi, Streptococcus pyogenes, Babesia microti, Mycoplasma pneumoniae, and/or Ehrlichia chaffeensis.

During initial primary herpesvirus infection, EBV, HCMV or HHV6 antibodies to multiple early, middle and late gene products, as well as those to complete virus particles, are produced, which may give rise to serum-specific IgM antibody titers to multiple gene products. Then, as the patient recovers, all serum antibody titers to early middle and late herpesvirus nonstructural genes disappear, and only positive serum antibody titers to complete structural virions, such as serum-specific IgG, remain.

In CFS, however, according to one aspect of the invention, there is “abortive” herpesvirus infection with no complete virion multiplication, and middle and late gene products may induce elevated serum antibody titers.

Therefore, in EBV CFS, there may be elevated serum antibody titers to EBV early, middle and late gene products not ordinarily present. In patients with HCMV CFS, there can be elevated serum antibody titers to HCMV early, middle and late gene products not ordinarily present. In patients with HHV6 CFS, there are elevated serum antibody titers to HHV6 early, middle and late gene products that are unique to CFS. In Group A CFS in which abortive infection by two or three of EBV, HCMV, HHV6 is present, appropriate dual or treble herpesvirus elevated early, middle and late serum antibody titers to gene products may be found. Abortive infection may be proven by, for example, finding evidence of appropriate mRNA molecules in circulating white blood cells in serum of the CFS patient.

An aspect of the invention, then, specifically indicates the presence of herpesvirus (whether EBV, HCMV, and/or HHV6) CFS by recognition of their individual early, middle or late messenger RNA in blood, and, in turn, specific serum antibody titers to these specific involved herpesviruses. Thus, according to an aspect of the invention, with a single specimen of blood from a CFS patient, the herpesviruses (whether EBV, HCMV, and/or HHV6) would be recognized and specific therapy could be immediately begun. This was not possible previously.

The detection of mRNA by nucleic acid based amplification (NASBA) is both highly specific and sensitive. mRNA is more sensitive for HCMV replication than HCMV antigenemia or HCMV DNA in leukocytes (leuko DNAemia).

Methods of Treatment

In another aspect, the invention provides methods of treating patients with CFS. In some embodiments, the methods of treating a patient with CFS involve evaluating the patient for serologic evidence of the presence of nucleic acid molecules that indicate primary infection by one or more CFS-causing agents, thereby detecting the presence of each CFS-causing agent present in the patient; evaluating the patient for serologic evidence of one or more co-infections; determining whether one or more co-infections are present in the patient; administering, or causing to be administered, to the patient a therapeutically effective amount of at least one pharmaceutical composition, further comprising at least one antiviral agent, such that each CFS-causing agent found in the patient is effectively treated by at least one antiviral agent administered to the patient; and, if one or more co-infections are present, also administering, or causing to be administered, to the patient a therapeutically effective amount of at least one pharmaceutical composition such that each co-infection found in the patient is effectively treated by at least one pharmaceutical composition administered to the patient, thereby treating the CFS. Each pharmaceutical composition can comprise one active agent, or it can comprise a cocktail of more than one active agent. The co-infection can be infection with, for example, Borrelia burgdorferi, Streptococcus pyogenes, Ehrlichia chaffeensis, Babesia microti, Mycoplasma pneumoniae, or a combination thereof. The antiviral agent can be, for example, valacyclovir, valganciclovir, maribavir, foscarnet, or a combination thereof.

Still a further aspect of the invention relates to methods of treating CFS in a patient, comprising: identifying expression of herpesvirus genes in an abortive infection process in a tissue of the patient, whereby one or more middle genes are expressed, but no complete virus is formed, and treating patients in whom these conditions are present with an antiviral agent effective to block the abortive infection. Identifying expression of herpesvirus genes can be done by detecting mRNA of the gene. Identifying expression of herpesvirus genes can also be done by immunoassay, which detects antibodies to the protein products of the gene.

In some embodiments, the invention provides methods for treating CFS by subclassifying CFS patients into two groups: patients with persistent abortive infection with Epstein-Barr virus (EBV), human cytomegalovirus (HCMV) and/or human herpesvirus 6 (HHV6), with no additional co-infections (referred to herein as Group A); and patients with persistent abortive EBV, HCMV, and/or HHV6 infection with co-infections by another agent. Such co-infections include, for example, Lyme disease, babesiosis, adult rheumatic fever, and mycoplasma myocarditis (referred to herein as Group B). These two groups of CFS patients meet the same international and CDC diagnosis criteria and previously have not been distinguished. Patients with such a co-infection do not respond well to solely antiviral treatment. The co-infection must be diagnosed and appropriately treated, in addition to treatment for the viral infections that may be present.

In still another aspect, the invention relates to methods of treating CFS comprising administering to a patient maribavir (MBV) (1263 W94, 1H-β-L-ribofuranoside-2-isopropylamino-5,6-dichlorobenzimidazole) and other antivirally effective benzimidazole derivatives. Maribavir and other benzimidazole derivatives are effective in treating, for example, CFS involving EBV and/or HCMV infection.

The compositions and methods of the present invention relate to the administration of, for example, valacyclovir, valganciclovir, maribavir, and/or foscarnet, and pharmaceutically acceptable salts, prodrugs, metabolites, analogues, derivatives and polymorphs thereof. In yet another aspect, the invention also relates to kits comprising any embodiment of the compositions of the present invention. For example, the invention contemplates a first pharmaceutical agent to treat a primary infection and a second pharmaceutical agent to treat a secondary infection, whether such agents are administered separately or in combined dosage forms.

In some embodiments, the methods of the invention involve evaluating the patient for serologic evidence of the presence of nucleic acid molecules that indicate the presence of one or more CFS-causing agents; administering to the patient a therapeutically effective amount of at least one pharmaceutical composition, further comprising at least one antiviral agent, such that each primary infection found in the patient is effectively treated by at least one antiviral agent administered to the patient, thereby treating the CFS. The CFS-causing agents can be, for example, EBV, HCMV, HHV6, or a combination thereof. The antiviral agent can be, for example, valacyclovir, valganciclovir, maribavir, foscarnet, or a combination thereof, though any agent that is effective against a CFS-causing agent can be used. According to these methods, the presence of CFS-causing agents can be detected before they would be detectable by the means currently known in the art, for example by the presence of antibodies to herpesvirus gene products. The serologic evidence of the presence of nucleic acid molecules permits a precise determination of which of the one or more CFS-causing agents is present. For example, the serologic evidence of the presence of specific nucleic acid molecules permits differentiation between HCMV infection and HHV6 infection. The nucleic acid molecules can be, for example, messenger RNA (mRNA) molecules. Messenger RNA specific to each herpesvirus precedes the presence of isotypic serum antibody. The mRNA is present within cells and can be detected as described herein.

Group A comprises CFS patients with EBV, HCMV, and/or HHV6 persistent infection in single virus or combination, but without additional co-infections. The following are the criteria for selecting these patients:

1) patients meet international and CDC criteria for CFS and have abnormal 24 Hr. ECG monitors (as determined by presence of tachycardia and/or abnormal T waves).

2) patients are positive for HCMV, EBV and/or HHV6 infection, as determined by detection of serum antibodies to each of these viruses or to gene products of these viruses, whether detected using enzyme-linked immunosorbent assay (ELISA) or other methods, including those of the invention.

3) patients are negative for CFS co-infections, such as, for example, infection by Borrelia burgdorferi, Streptococcus pyogenes, Ehrlichia chaffeensis, Babesia microti and Mycoplasma pneumoniae. These patients respond favorably, their validated Energy Index Point scores increase from ≦5 to ≧6, usually reaching an Energy Index Point Score (EI) of 7-9 within 6 to 12 months of antiviral therapy with appropriate antiviral agents, including, for example, valacyclovir, valganciclovir, and maribavir.

Group B comprises CFS patients with EBV, HCMV, and/or HHV6 persistent infection, either singly or in combination, but also having one or more additional co-infections. The following are the criteria for Group B patients:

1) patients meet international and CDC criteria for CFS and have abnormal 24 Hr. ECG monitors (as determined by presence of tachycardia and/or abnormal T waves).

2) patients are positive for HCMV, EBV and/or HHV6 infection.

3) patients are positive for one or more of the following: Borrelia burgorferi, Streptococcus pyogenes, Ehrlichia chaffeensis, Babasia microti, and Mycoplasma pneumoniae.

CFS patients in Group B can be treated with, for example, valacyclovir, valganciclovir, or maribavir or other derivatives or benzimidazole, as appropriate to the particular infections present in a given patient. CFS patients in Group B can also be treated with appropriate therapy for co-infection, which may include, for example, infection with Borrelia burgorferi, Streptococcus pyogenes, Ehrlichia chaffeensis, Babesia microti, and Mycoplasma pneumoniae, optionally prior to antiviral therapy. Generally, unless Group B patients are treated for the co-infection they will not improve. As used herein, “improve” means to reduce the severity of, or eliminate, one or more of the symptoms of CFS.

Reference serologic studies for detection of Lyme disease, Babesiosis, Ehrlichiosis, and Mycoplasma pneumoniae are available from, for example, LabCorp, Dublin, Ohio.

Infection with Borrelia burgdorferi can be diagnosed by, for example, detecting the presence of IgM or IgG to Borrelia burgdorferi using Western blot or ELISA. It may be possible that IgG is not detected in a patient with CFS, in which case a positive IgM result would be diagnostic of infection with Borrelia burgdorferi. Antigens used for this assay are exact prototypes used by US Centers for Disease Control.

Infection with Borrelia burgdorferi can be treated with, for example, intravenous (IV) ceftriaxone 0.1-5 gm, for example 1.0-1.5 gm, intravenous piggy-back (IVPB) every 12 hours for 30 days, followed by oral amoxicillin 0.01-4.0 gm, for example 0.5-0.75 gm, 4 times/day until above serum tests negative. IV penicillin G or its equivalent every 6-8 hours can substitute for ceftriaxone. For treatment of Borrelia burgdorferi, as well as of any other secondary infection described herein, qualified health care personnel can prescribe appropriate dosages for effective treatment of CFS. Any dosage that falls within the scope of sound medical judgment is contemplated as part of this invention.

Adult rheumatic fever is caused by a hyperimmune response to, for example, Streptococcus pyogenes infection. It can be diagnosed by, for example, finding an elevated antistreptolysin O (ASO) titer (LabCorp, Dublin, Ohio); for example, an ASO titer over 400 units would be diagnostic of adult rheumatic fever or co-infection by Streptcoccus pyogenes. Adult rheumatic fever may also be accompanied by, for example, thickening of the aortic and/or mitral valve, which can be viewed on an echocardiogram.

Adult rheumatic fever can be treated with, for example, ceftriaxone or penicillin G as above followed by bicillin 0.1-5 Mu, for example 1.2 Mu, every 2-4 weeks until ASO titer is less than 200. A CatScan of sinuses/mastoids may be indicated to exclude obstructive sinusitis. Bicillin may be necessary for 2-4 years.

Babesiosis can be diagnosed by, for example, finding an elevated serum titer for IgG to Babesia microti. Chronic Babesiosis can be treated with, for example, Ataquavone 100-1500, for example 750 mg orally, plus azithromycin 0.01-4 gm, for example 0.5 gm, twice daily for 6 weeks. Detection of IgM to Babesia microti can also be done.

Ehrlichia chaffeensis co-infection can be determined by, for example, detecting a positive serum titer for IgG or IgM to Ehrlichia chaffeensis. Chronic Ehrlichiosis can be treated with, for example, IV doxycycline 10-1000 mg, for example 100 mg, every 12 hours for 4, 6, or 12 weeks.

Infection by Mycoplasma pneumoniae can be diagnosed by, for example, finding a markedly positive serum titer for IgG or IgM to Mycoplasma pneumoniae. Infection by Mycoplasma pneumoniae may also be accompanied by, for example, an abnormal standard 12-lead electrocardiogram.

Mycoplasma pneumoniae myocarditis can be treated with, for example, IV doxycycline plus/minus IV azithromycin for 6 weeks.

Effective sublassifications of CFS patients have not been previously recognized. The failure to recognize the variety of infections that may afflict CFS patients, and thus the failure to segregate them when conducting placebo-controlled randomized trials, has resulted in tested antiviral therapies falsely appearing ineffective. As a result, CFS patients have gone without effective treatment.

Methods of Preventing

In another aspect, the invention provides methods of preventing CFS in a patient, comprising diagnosing the patient according to the methods described above; detecting the presence of nucleic acid molecules that are indicative of infection by one or more CFS-causing agents, thereby detecting the presence of each CFS-causing agent present in the patient; and administering to the patient a therapeutically effective amount of a pharmaceutical composition comprising at least one antiviral agent, such that each CFS-causing agent detected in the patient is effectively treated by at least one antiviral agent administered to the patient, thereby treating the CFS.

Kits

In yet another aspect, the invention provides kits. The kit can comprise, for example, means for detecting serologic evidence of HCMV; means for detecting serologic evidence of EBV; means for detecting serologic evidence of HHV6; and means for detecting serologic evidence of at least one non-viral pathogen selected from the group consisting of Borrelia burgdorferi, Streptococcus pyogenes, Ehrlichia chaffeensis, Babesia microti, and Mycoplasma pneumoniae. The means for detecting serologic evidence of any of these pathogens can be any means indicated herein. A particular means may be effective to detect serologic evidence of more than one pathogen.

### Pharmaceutical Compositions and Administration

Benzimidazole ribosides and their derivatives, such as, for example, maribavir, offer potent new therapeutic options for CFS patients, in addition to such antiviral agents as, for example, valacyclovir, valganciclovir and foscarnet—all of which inhibit the DNA polymerase enzyme thymidine kinase in the herpesviruses, and which may have major human toxicities affecting the bone marrow or liver or which may cause leukopenia, thrombocytopenia or cancer. Maribavir inhibits HCMV multiplication by inhibiting UL97 and viral assembly of HCMV. The only side effects of maribavir administration have been changes in taste and headache, and then only in high dose repetitive human studies. Maribavir also inhibits EBV processivity factor EA-D and can be used in the therapy of EBV CFS and HCMV CFS disease as well. Maribavir can be delivered orally in doses of, for example, from about 100 mg to about 1600 mg up to four times per day. For example, maribavir can be delivered in doses of up to or at least about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1400 mg, about 1500 mg, about 1600 mg or more, up to four times per day. It is excreted by the hepatobiliary circulation and no adjustment for renal dysfunction is needed. The 50% inhibitory concentration of maribavir versus HCMV is 0.12±0.01 μM compared to 0.53±0.04 μM for ganciclovir.

The antiviral agent, whether maribavir or otherwise, can be administered by any appropriate method, including oral, rectal, nasal, topical, vaginal and parenteral, including subcutaneous, intramuscular, intravenous, intradermal, intrathecal, and epidural.

In general, to provide a therapeutically effective amount of the antiviral agent, a suitable effective dose will be in the range of 0.1 to 20 grams per day and preferably in the range between 0.3 and 15 grams per day, more preferably about 0.5 to 10 grams per day. The dosage, of course, varies with the body weight of the patient and so for a 70 kg individual, a dose of 4 grams per day may be appropriate (e.g., 10 mg per kg valacyclovir every 6 hours). The desired dose can be presented as 2, 3, 4 or more smaller doses administered at appropriate intervals throughout the day. These smaller doses may be administered in unit dosage forms. For example, for valacyclovir and famciclovir, the dosage can be, for example, 14 mg/kg every 6 hours (1.0 g every 6 hours for a 70 kg person). The dosage of valacyclovir and famciclovir can be, for example, up to or at least about 0.5, about 1.0, about 1.5, about 2.0, about 2.5, about 3, about 3.5, about 4, about 4.5, about 5, about 5.5, about 6, about 6.5, about 7, about 7.5, about 8 or more grams every 6 hours. The dosage of valgancyclovir can be, for example, from about 450 to about 900 mg every 12 hours, or up to or at least about 100, about 200, about 300, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000 mg or more every 12 hours, depending on, for example, patient weight and tolerance. For maribavir the dosage can be, for example, from about 400 to about 500 mg every 8 hours, or for example up to or at least about 100, about 150, about 200, about 250, about 300, about 350, about 400, about 450, about 500, about 550, about 600, about 650, about 700, about 750, about 800, about 900, about 1000 or more every 8 hours. Qualified health care personnel can prescribe appropriate dosages for effective treatment of CFS. Any dosage that falls within the scope of sound medical judgment is contemplated as part of this invention.

In particular for valacyclovir, or a derivative such as valacyclovir hydrochloride, a patient can be administered a dosage in the range of 0.1 to 50 mg/kg body weight of the patient per dosing interval, generally every six hours. The dosing interval is determined by the bioavailability of the antiviral agent and its excretion from the body. For example, the patient can be administered a dosage in the range of 0.3 to 40 mg/kg of body weight of valacyclovir hydrochloride orally every six hours. For example, a patient can be administered 10 mg/kg of body weight of valacyclovir hydrochloride every six hours.

The treatment period for a CFS patient varies on a case-by-case basis. It is believed that for some, CFS is an ongoing and persistent problem requiring continued treatment. The duration of the therapy depends on the intensity of the CFS as affected by the therapy. One indicator of an improvement in EBV-isolated CFS patients is a decrease in the level of IgM antibodies to viral capsid antibodies (VCA) for EBV. Generally, the therapy duration is proportional to the intensity of the CFS manifestation. Accordingly, following administration of an anti-viral agent, supplemental tests are helpful to check for recurrent CFS and to determine the treatment duration. The duration of treatment may be 6 months, 9 months, 12 months, 18 months, or longer or shorter than any of these periods, as may be determined by a physician using the methods of the invention.

Antiviral agents which demonstrate anti-herpetic action, such as those specific to, for example, EBV, HCMV or HHV6, can be used for the treatment of chronic fatigue syndrome. Such antiviral agents may be effectively administered, for example by oral methods, or as larger doses in time delay formulations. Included among this group of antiviral agents are acyclovir, valacyclovir, valganciclovir, famciclovir, maribavir, and other herpetic antiviral agents and pharmaceutically acceptable derivatives of these antiviral agents. Such pharmaceutically acceptable derivatives include salts, hydrolyzable esters and chelates of the antiviral agents and such similar derivatives which have no negative pharmaceutical effect on the patient upon administration and are thus “pharmaceutically acceptable.” A pharmaceutically acceptable salt can be, for example, an acidic salt derived from an appropriate acid, for example, hydrochloric, sulfuric, phosphoric, maleic, fumaric, citric, tartaric, lactic, acetic or p-toluenesulphonic acid.

### EXAMPLES

### Example 1

### Subset Directed Antiviral Therapy of Cytomegalovirus, Epstein-Barr Virus Co-Infection Chronic Fatigue Syndrome

Ten patients with chronic fatigue syndrome (CFS) of 1.3-2.6 years duration had elevated serum antibody titers to cytomegalovirus (HCMV), and 9 of these patients also had elevated serum antibody titers to Epstein-Barr virus (EBV). They were classified: HCMV-EBV subset (nine patients) and HCMV subset (one patient) CFS. We determined whether subset classification is required for successful antiviral therapy of CFS.

CFS patients with elevated serum antibody titers to HCMV and EBV were treated with valganciclovir (HCMV) and valacyclovir (EBV) according to subset classification for a mean of 2.6±0.4 years. The 10 patients returned to health and remain well. Five patients continue no antiviral drug, and five patients require continuing antiviral suppressive therapy. No significant side effects were observed. Subset directed antiviral therapy leads to sustained recovery in CFS patients.

These methods are used, according to the invention, in conjunction with assays for other infections, including, for example, those used to identify patients with HHV6 and/or secondary infection. Patients may thus be stratified and monitored for efficacy of treatment using the serological methods, including gene expression analysis, according to the invention.

### Example 2

### Immunoassay with Cytomegalovirus Early Antigens from Gene Products p52 (UL44) and CM₂ (UL44 and UL57) Detect Active Infection in Patients with Chronic Fatigue Syndrome

The purpose of this study was to demonstrate that use of recombinant early antigens for detection of antibodies to cytomegalovirus (HCMV) gene products CM₂ (UL44, UL57) and p52 (UL44) is specific in the diagnosis and differentiation of active HCMV infection in a subset of patients with chronic fatigue syndrome (CFS) which is often missed by current ELISA assay that uses crude viral lysate antigen. At a single clinic a total of 4,774 serologic tests were performed in 1135 CFS patients using two immunoassays: Copalis immunoassay and ELISA immunoassay. The Copalis immunoassay utilized HCMV early gene products of UL44 and UL57 recombinant antigens for detection of HCMV IgM antibody and viral capsid antigen for detection of HCMV IgG antibody The ELISA immunoassay utilized viral crude lysate as antigen for detection of both HCMV IgG and IgM.

Of the total 1135 suspected CFS patients, 517 patients (45.6%) were positive for HCMV IgG by both assays. Of these, twelve CFS patients (2.2%) were positive for HCMV IgM serum antibody by ELISA assay, and 61 CFS patients (11.8%) were positive for IgM serum antibody by Copalis assays. The Copalis assay that uses HCMV early recombinant gene products CM₂ (UL44, UL57) and p52 (UL44) in comparison with ELISA was >98% specific. Immunoassays that use recombinant CM₂ and p57 (UL44 and UL57) HCMV gene products as an antigen are five times more sensitive than ELISA assay, and are specific in the detection and differentiation of active HCMV infection in a subset of CFS patients.

These methods are used, according to the invention, in conjunction with assays for other infections, including, for example, those used to identify patients with HHV6 and/or secondary infection. Patients may thus be stratified and monitored for efficacy of treatment using the serological methods, including gene expression analysis, according to the invention.

### Example 3

### Valacyclovir Treatment in Epstein-Barr Virus Subset Chronic Fatigue Syndrome, Thirty-Six Month Follow-up

We used subset classification of Epstein-Barr virus (EBV) in chronic fatigue (CFS). We (1) performed (Group 1) a blinded-random placebo-controlled trial of valacyclovir in EBV CFS subset, and (2) followed (Group 2) this EBV subset for thirty-six months. Patients were given valacyclovir 14.3 mg per kg every 6 hours. The validated Energy Index point score (EI) assessing physical functional capacity, Holter monitor, multigated (radionuclide) MUGA rest/stress ventriculographic examination, EBV serum IgM viral capsid antibodies (VCA), and EBV Early Antigen diffuse, (EA) were evaluated.

Group 1: after six months CFS patients receiving valacyclovir experienced an increased mean least square EI Point Scores +1.12 units (122 Kcal/day), while the placebo cohort increased +0.42 EI units (+65 Kcal/day). Group 2: EI Point Scores increased progressively. Sinus tachycardias decreased and abnormal cardiac wall motion improved. Serum antibody titers to EBV VCA IgM decreased. Patients resumed normal activities of living.

These methods are used, according to the invention, in conjunction with assays for other infections, including, for example, those used to identify patients with HHV6 and/or secondary infection. Patients may thus be stratified and monitored for efficacy of treatment using the serological methods, including gene expression analysis, according to the invention.

### Example 4

### Treatment of Herpesvirus CFS with Antiviral Nucleosides

Long-term benefits were found for 90 of 124 (72.6%) of CFS patients treated at a single clinic. The antiviral medicines, as administered, were safe.

Methods

Background and Patient Population The international criteria for diagnosis of CFS was used. With strict confidentiality clinical files of CFS patients were identified by number only. A blinded code was maintained. Data were entered to the Axel computer system and analyzed anonymously. Patients with active EBV infection were identified by the presence of elevated serum ELISA antibodies to Epstein-Barr virus (EBV) IgM viral capsid antigen p18 peptide (Diasorin Inc., Stillwater, Minn.) and/or EBV, Early antigen (diffuse) 47 KD recombinant peptide (Diasorin, Stillwater, Minn.). Patients with active HCMV infection were identified by the presence of HCMV strain AD69 human fibroblast lysate, IgM and IgG ELISA and Copalis light-scatter, HCMV IgM p52 recombinant protein, UL44 and HCMV CM2, UL44 and part UL57 recombinant protein. Elevated IgM and IgG Human Herpesvirus 6 serum antibody titers ≧160 (LabCorp, Dublin, Ohio) were recorded. CFS patients had 24-Hr ECG Holter monitor recordings. When abnormal resting standard ECG and abnormal recurrent oscillating T-waves (flat, inverted) were present at baseline, radionuclide ventriculography by bicycle protocol stress studies were performed. The CFS patients reported here are termed Group A. They had negative IgM, IgG serum antibody titers to Borrelia burgdorferi, Western blot and ELISA; anti-streptolysin 0 titers <200 units; and negative serum IgM and IgG antibody titers to both Ehrlichia chaffeensis and Babesia microti (LabCorp).

Measurement of Degree of Fatigue (Physical Capacity) The Energy Index Point Score (EIPS) is a reliable validated metric assessing the degree of illness of CFS patients. With an EIPS scoring scale for reference on the wall in each examining room, the EIPS was determined by consensus of physician and patient. An EIPS of “0” is a bedridden patient. Patients were seen every 4-6 weeks and EIPS recorded. If an intercurrent illness was present EIPS determinations were delayed for 2 weeks. An EIPS of between 0 and 5 is associated with the presence of CFS. At an EIPS of between 6 and 10, the patient no longer meets criteria for diagnosis of CFS. A change in EIPS of 0.25 is small, a 0.5 change in EIPS is a medium change, and an EIPS change of ≧0.8 is a large effect size (p<0.05).

Valacyclovir and Famciclovir Administration to CFS patients with EBV infection Valacyclovir and famciclovir are pro-drugs well absorbed orally and, after reaching the circulation as acyclovir or penciclovir, respectively, inhibit EBV thymidine kinase efficiently. The infectious dose-50 in tissue culture (ID₅₀) for both agents is 1.1-4.4 μg/ml. Here, valacyclovir was the agent of first choice, but when diarrheas to valacyclovir developed, famciclovir was substituted. Valacyclovir and famciclovir were given in 1 gm doses to 70 kg patients every 6 hours reaching peak acyclovir levels at 90-120 min. of 7.9-21 μg/ml. If the patient weighed over 79.5 Kg, 1500 mg valacyclovir was given at each dose. Valacyclovir is excreted by the kidney by both glomerular filtration and tubular secretion. Valacyclovir renal calculi may result, and patients were advised to drink at least 1500 ml water daily. The T ½ of both acyclovir and penciclovir is 1.5-6 hours. Valganciclovir and famciclovir are not effective therapeutic agents for HCMV or HHV6. At physician discretion cimetidine (400 mg every 12 hours) and/or probenecid (500 mg every 12 hours), which inhibit the tubular secretion of valacyclovir, were given to increase acyclovir serum levels. Valacyclovir, once absorbed orally, is acyclovir in serum.

Valganciclovir Administration to CFS patients with HCMV and/or HHV6 infection The ID₅₀ of valganciclovir for HCMV and HHV6 is 0.2-2.8 μg/ml., but in simultaneous tests, valganciclovir is one-tenth as active versus EBV as compared to HCMV. Therefore, valganciclovir was employed in CFS associated with HCMV or HHV6. Valganciclovir is efficiently absorbed as ganciclovir. It is inhibitory to human bone marrow progenitor cells and lymphocyte blastogenesis at this dosage. After 0.5-1.0 gm doses, the average peak concentrations (Cₘₐₓ) is 6.1 μg/ml. Food increases the bioavailability of both valacyclovir and valganciclovir.

CFS patients were given valganciclovir. Cautiously beginning, 450 mg daily was administered every morning after a meal for three days, and then increasing dosage to two doses of 450 mg, every morning for three days. They were then given two doses of 450 mg valganciclovir every morning followed by one 12 hours later as a continuing dosage. If any toxicity was observed at 4-6 week visits, valganciclovir was held for one to two weeks and then continued as 900 mg every 24 hrs. If the patient weighed over 90.0 Kg, 900 mg valganciclovir every 12 hours was given. Dosage was evaluated every 2-6 weeks for ongoing tolerance with normal values of hemograms, platelets and amino transferases and creatinines.

Atenolol and fludrocortisones acetate At baseline, syncope with marked heart rate variability, and tachycardias at rest were usually present. Atenolol 12.5-50 mg every 12 to 24 hours was given at baseline to control tachycardias. Twenty-four hour ECG monitors were repeated seven days after beginning atenolol to monitor response. One-tenth mg fludrocortisones acetate every 12 to 24 hours was given for syncope and orthostatic hypotension. With improvement in the EIPS to 7, atenolol and fludrocortisones were tapered and discontinued.

Toxicity of Antiviral Nucleosides Patients were seen every 4 to 6 weeks, EIPS was recorded and complete blood count, aminotransferases, creatinine and urinalyses were done. Also recorded were abnormalities in white blood cells and platelet counts, aminotransferases, weight gain or loss, whether famciclovir was substituted for valacyclovir, dosage change for valganciclovir, valacyclovir, or famciclovir, and whether any of the following occurred: participation in blinded-placebo-controlled study, hospitalization, surgery, recovery, a cessation of medicines by patient choice, cessation of medicines by physician choice, relapse with worsening EIPS and returned for care.

Results

Demographics (Table 3) There were 124 CFS patients (75.8% females, 24.2% males) and elevated serum antibody titers to EBV, HCMV, and HHV6 in single or co-infection who were treated in this clinic study for 6 months or longer. They had no elevated serum antibody titers to Babesia microti, Borrelia burgdorferi, or Ehrlichia chaffeensis. Antistreptolysin 0 titers were <400. These CFS patients we classified as having Group A CFS. The Group A CFS patients' mean age was 46.4±1.3 years and BMI was 26.6±5.2 Kg/m². The mean duration of CFS illness prior to receiving any antiviral nucleosides was 4.9 years.

TABLE 3
Demographics of 124 Group A Herpesvirus Chronic Fatigue
Syndrome (CFS) Patients Treated with Antiviral Nucleosides
According to Subset Classification at a Single Clinic.

| | |
|---|---|
| Females | 94 pts. (75.8%) |
| Males | 30 pts. (24.%) |
| Age | 46.4 ± 1.3 yrs.* |
| BMI | 26.6 ± 5.2 Kg/m²* |
| Mean duration of illness prior to antiviral | 4.9 + 5.3 yrs* |
| therapy | |
| Mean duration of antiviral therapy | 3.2 years |
| EIPS (energy index point score), baseline | 4.19 ± 0.87 (4.34 ± 0.81 for |
| | 90 CFS pt. “responders”) |
| EIPS (energy index point score), last | 5.61 ± 1.89 (6.50 ± 1.40 for |
| | 90 CFS pt. “responders”) |
| EIPSₗₐₛₜminus EIPS_{baseline} | +1.8 (+2.2 for “responders”) |

*Standard deviation of mean
+There were 63 Group B Herpesvirus patients, 2001-2007. Group B CFS patients have herpesvirus infections similar to Group A with additionally co-infections: Lyme disease, Babesiosis, Ehrlichiosis, adult Rheumatic Fever or Mycoplasma pneumoniae myocarditis.

Herpesvirus Subset Classification of Group A CFS Patients (Table 4) Single herpesvirus Group A CFS patients comprised 48 of the 124 (38.7%) Group A patients. Of these, 31 (25%) were single EBV subset; 15 (12.1%) were single HCMV subset; and 2 (1.6%) were single HHV6 subset. The mean age of CFS patients with herpes virus single virus subset was 46.4 years: they were 68.8% female and their mean BMI was 27.2±7.1 Kg/m². Seventy-six (61.3%) Group A CFS patients exhibited multiple herpesvirus infection. Their mean age was 46.8 years, and 62 (81.6%) were females and 14 (18.4%) males. The mean BMI for the multi-herpes co-infection subsets was 27±2.2. There were 38 (30.6%) CFS patients in the EBV/HHV6 co-infection subset; 7 (5.6%) CFS patients in the HCMV/HHV6 co-infection subset; 7 (5.6%) CFS patients in the HCMV/HHV6 co-infection subset; and 17 (13.7%) CFS patients in the EBV/HCMV/HHV6 co-infection subset.

TABLE 4
Herpesvirus Subset Classification of 124 Group A CFS Patients

| | No. of Pts | | Age (yrs) | Females | | Males | | B.M.I. |
|---|---|---|---|---|---|---|---|---|
| Single Herpesvirus Subsets | | | | | | | | |
| a) Epstein-Barr virus (EBV) | 31 | (25%) | 42 ± 14* | 21 | (67.7%) | 10 | (32.3%) | 26.7 ± 4.4 |
| b) Cytomegalovirus (HCMV) | 15 | (12.1%) | 44.5 ± 15.2 | 10 | (66.7%) | 5 | (33.3%) | 25.4 ± 4.7 |
| c) Human Herpesvirus 6 (HHV6) | 2 | (1.6%) | 44 ± 19.8 | 2 | (100%) | 0 | (0%) | 29.4 ± 12.1 |
| Total single-virus subsets | 48 | (38.7%) | 46.4 ± 13.2 | 33 | (68.8%) | 15 | (31.2%) | 27.2 ± 7.1 |
| Multiple Herpesvirus Subsets | | | | | | | | |
| a) HBV/HCMV | 38 | (30.6%) | 49.7 ± 12.9 | 31 | (81.6%) | 7 | (18.4%) | 27.4 ± 6 |
| b) EBV/HHV6 | 14 | (11.3%) | 42.9 ± 12.7 | 11 | (78.6%) | 3 | (21.4%) | 24.8 ± 5.0 |
| c) HCMV/HHV6 | 7 | (5.6%) | 42.9 ± 10.5 | 7 | (100%) | 0 | (0%) | 29.7 ± 6.5 |
| d) EBV/HCMV/HHV6 | 17 | (13.7%) | 51.7 ± 8.4 | 13 | (76.5%) | 4 | (23.5%) | 26.0 ± 3.4 |
| Total Multiple herpesvirus | 76 | (61.3%) | 46.8 ± 4.7 | 62 | (81.6%) | 14 | (18.4%) | 27.0 ± 2.2 |
| subsets | | | | | | | | |

*Standard error of the mean.

Therefore, according to these Group A CFS data, and combining both single and multiple infections, 40 CFS patients had HHV6 infections; 77 CFS patients had HCMV infections; and 100 CFS patients had EBV infections.

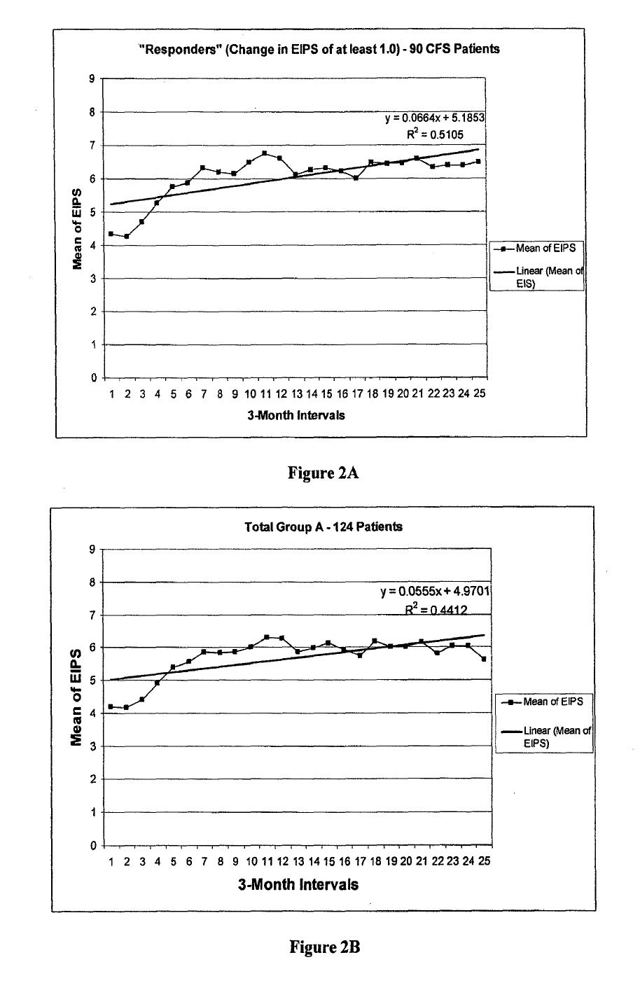

Increases in Energy Index Point Score (EIPS) Among Patients After Receiving Antiviral Nucleosides, Group A CFS (Table 5, FIG. 2A) The baseline mean EIPS for entire Group A CFS patients was 4.19, while the mean final EIPS five years later was 5.61, a large effect mean increase of 1.4 EIPS units. The mean duration of antiviral therapy was 3.2 years. Changes in mean EIPS for the entire group of 124 CFS Group A patients, measured at 3 month intervals from baseline starting in 2001 and continuing into 2007, are presented in Table 5 and FIG. 2A. For the “responders,” who experienced a positive EIPS change of 0.8, the baseline EIPS was 4.34. A patient was classified as a “responder” if he or she experienced an EIPS increase of 1.0 or more After beginning antiviral nucleosides both responders and non-responders felt increased fatigue for two to six weeks. At one year of antiviral nucleoside therapy, the 83 CFS patients who were evaluated and had achieved “responder” status by this point had a mean EIPS of 5.75 at one year; the 61 responders evaluated after two years of treatment had a mean EIPS of 6.14. CFS patients maintained or increased their EIPS for the remaining four years of the study. The mean EIPS at 24 months for the responders was 6.50. The probability of these data occurring by chance is <0.002. There is an average of 46 CFS patients for each 3 month interval mean EIPS of the 24 months.

TABLE 5
Comparisons of EIPS Scores at Three Month Intervals in CFS Patients (Total
124 CFS Group A Patients Versus Responders (90 Patients, Change EIPS ≧ 1.0)

| GROUP A “RESPONDERS” - 90 PATIENTS | | | | TOTAL GROUP A - 124 PATIENTS | | | |
|---|---|---|---|---|---|---|---|
| | | | D | | | | H |
| A | B | C | Standard | E | F | G | Standard |
| 3 Month | Number of | Mean of | Deviation | 3 Month | Number of | Mean of | Deviation |
| Intervals | Patients | EIPS | of EIPS | Intervals | Patients | EIPS | of EIPS |
| 0 | 90 | 4.34 | 0.81 | 0 | 124 | 4.19 | 0.87 |
| 1 | 86 | 4.27 | 0.94 | 1 | 116 | 4.18 | 0.95 |
| 2 | 87 | 4.70 | 1.18 | 2 | 121 | 4.42 | 1.19 |
| 3 | 86 | 5.26 | 1.46 | 3 | 120 | 4.90 | 1.47 |
| 4 | 83 | 5.75 | 1.41 | 4 | 112 | 5.39 | 1.50 |
| 5 | 77 | 5.88 | 1.41 | 5 | 99 | 5.57 | 1.50 |
| 6 | 75 | 6.32 | 1.30 | 6 | 95 | 5.86 | 1.52 |
| 7 | 67 | 6.19 | 1.32 | 7 | 83 | 5.84 | 1.51 |
| 8 | 61 | 6.14 | 1.34 | 8 | 72 | 5.85 | 1.52 |
| 9 | 56 | 6.48 | 1.06 | 9 | 67 | 6.02 | 1.50 |
| 10 | 53 | 6.75 | 1.19 | 10 | 62 | 6.31 | 1.57 |
| 11 | 52 | 6.61 | 1.22 | 11 | 62 | 6.28 | 1.49 |
| 12 | 46 | 6.11 | 1.45 | 12 | 54 | 5.87 | 1.60 |
| 13 | 44 | 6.27 | 1.43 | 13 | 51 | 5.98 | 1.60 |
| 14 | 46 | 6.31 | 1.48 | 14 | 53 | 6.12 | 1.55 |
| 15 | 40 | 6.23 | 1.33 | 15 | 46 | 5.93 | 1.53 |
| 16 | 37 | 6.00 | 1.58 | 16 | 42 | 5.73 | 1.72 |
| 17 | 35 | 6.49 | 1.50 | 17 | 41 | 6.19 | 1.69 |
| 18 | 35 | 6.45 | 1.42 | 18 | 42 | 6.02 | 1.64 |
| 19 | 29 | 6.47 | 1.40 | 19 | 36 | 6.00 | 1.68 |
| 20 | 26 | 6.59 | 1.65 | 20 | 33 | 6.15 | 1.82 |
| 21 | 21 | 6.34 | 1.45 | 21 | 28 | 5.81 | 1.69 |
| 22 | 21 | 6.39 | 1.11 | 22 | 26 | 6.04 | 1.41 |
| 23 | 19 | 6.38 | 1.23 | 23 | 24 | 6.03 | 1.46 |
| 24 | 12 | 6.50 | 1.40 | 24 | 17 | 5.61 | 1.89 |

These data are also shown in FIG. 2A. For the total of 90 Group A CFS responders 46 patients (37.1%) had mean increases in EIPS of between 1 and 3 units; 33 (26.5%) CFS patients had mean increases in EIPS of between 3 and 4 units; and 11 (8.8%) CFS patients had increases in EIPS of at least 4.

Increases in EIPS Among Group A CFS Patients Treated with Antiviral Nucleosides According to Subset (Table 6) CFS patients in the EBV subsets were treated with valacyclovir/famciclovir. CFS patients in the HCMV and HHV6 subsets were treated with valganciclovir. CFS patients in EBV subset with HHV6 and/or HCMV co-infection were treated with valganciclovir. Over the six year study, CFS patients in one of the single herpesvirus subsets increased their EIPS by a mean of 1.8 EIPS (EBV single-virus subset, +2; HCMV single-virus subset, +1.5; HHV6 single-virus subset, +2.7). Multiple herpesvirus subsets similarly experienced a mean increase of 1.7 EIPS (EBV/HCMV subset, +1.8; EBV/HHV6 subset, +2.0; HCMV/HHV6 subset, +1.7).

TABLE 6
Mean Increase in EIPS Among 124 Group A CFS Patients Treated
with Antiviral Nucleosides, According to Subset

| | | | Duration of | | | |
|---|---|---|---|---|---|---|
| | | | CFS Prior | | | |
| | | | to Antiviral | EIPS | | |
| | No. of Pts | | Therapy | Baseline | Last | Change |
| Single Herpesvirus Subsets | | | | | | |
| a) EBV | 31 | (25%) | 2.8 | 4.3 | 6.3 | +2 |
| b) HCMV | 15 | (12.1%) | 8 | 4.1 | 5.6 | +1.5 |
| c) HHV6 | 2 | (1.6%) | 2.3 | 4.3 | 7 | +2.7 |
| Total single-virus subsets | 48 | (38.7%) | 4.9 | 4.2 | 6.0 | +1.8 |
| Multiple Herpesvirus Subsets | | | | | | |
| a) EBV/HCMV | 38 | (30.6%) | 4.6 | 4.0 | 5.8 | +1.8 |
| b) EBV/HHV6 | 14 | (11.3%) | 4.2 | 4.4 | 6.4 | +2.0 |
| c) HCMV/HHV6 | 7 | (5.6%) | 4.8 | 3.9 | 5.0 | +1.1 |
| d) EBV/HCMV/HHV6 | 17 | (13.7%) | 7.1 | 4.5 | 6.2 | +1.7 |
| Total Multiple Herpesvirus | 76 | (61.3%) | 5.2 | 4.2 | 5.9 | +1.7 |
| Subsets | | | | | | |

Detail of the Magnitude of EIPS Increases Among Group A CFS Single and Multiple Herpesvirus Subsets. (FIG. 2B) The magnitude of the increase in EIPS between baseline and last EIPS is presented in FIG. 2B. For the EBV CFS single-virus subset patients, 23 of these 31 CFS patients (74.2%) are “responders” to antiviral nucleoside, with increases in EIPS of ≧1.0. One patient achieved a final EIPS of 9, 9 patients achieved a final EIPS of between 8 and 9, and 8 patients whose last EIPS was between 7 and 8.

For 15 CFS patients in the single virus subset, HCMV (66.7%). There were 2 HCMV subset patients whose last EIPS was at least 8, one HCMV subset patient whose last EIPS was between 7 and 8, and 6 HCMV subset patients whose last EIPS was between 6 and 7.

Among multiple Group A CFS subsets 26 EBV/HCMV patients were “responders” (68.4%). There were 6 CFS patients whose last EIPS was at least 8, 10 CFS patients whose last EIPS was between 7 and 8, and 7 CFS patients whose last EIPS was between 6 and 7.

Among the EBV/HHV6 CFS “responders” (85.7%) of this Group A subset, there were 3 patients with a last EIPS of 8; 3 patients with a last EIPS of between 7 and 8; and 5 patients with a last EIPS of between 6 and 7. Among the HCMV/HHV6 subset there were 42.8% “responders.” One CFS patient's last EIPS was 7 and one patient's last EIPS was 6.5. Among the EBV/HCMV/HHV6 subset, there were 82.4% “responders.” There was one patient whose last EIPS was 8.5; and 4 patients whose last EIPS was 8. There were 2 patients whose last EIPS was 7 and 4 patients whose last EIPS was between 6 and 7.

Toxicity There were no changes in hemoglobin or platelet levels in patients receiving valganciclovir, though increases in aminotransferases were observed. With modifications in valganciclovir dosing as outlined herein, no patient, after tolerating the initial weeks of worsening symptoms at the beginning of valacyclovir, famciclovir or valganciclovir administration, had to stop therapy. There was no toxicity due to valacyclovir or famciclovir.

### Example 5

### Diagnostic Panel For Group A and Group B CFS

Determination of the etiology of CFS in a patient can be done using the following diagnostic panels, divided into Group A and Group B panels. Each assay is an example of a means for detecting serologic evidence of the associated pathogen.

TABLE 7
CFS Group A Diagnostic Panel - Antibody Assays

| Pathogen | Assay Method/Target |
|---|---|
| EBV | EBV, VCA, IgM-antigen-VCA p18 peptide |
| | EBV, EA(D) - 47KD recombinant peptide |
| HCMV | HCMV (Strain AD 69, human fibroblast lysate), IgM |
| | HCMV (Strain AD 69, human fibroblast lysate), IgG |
| | HCMV p52, recombinant protein, UL44 |
| | HCMV CM2, UL44 and part UL57 recombinant protein |
| HHV6 | HHV6, IgM |
| | HHV6, IgG |

TABLE 8
CFS Group B Diagnostic Panel

| | Associated | |
|---|---|---|
| Pathogen | disease | Assay Method/Target |
| Streptococcus | Adult | Antistreptolysin O |
| pyogenes | rheumatic fever | |
| Borrelia burgdorferi | Lyme disease | Western blot, IgM |
| | | Western blot, IgG |
| Ehrlichia chaffeensis | Ehrlichiosis | Ehrlichia chaffensis, IgM |
| | | Ehrlichia chaffensis, IgG |
| Babesia microti | Babesiosis | Babesia microti, IgM |
| | | Babesiia microti, IgG |
| Mycoplasma | Mycoplasma | Mycoplasma pneumoniae, IgM |
| pneumoniae | myocarditis | Mycoplasma pneumoniae, IgG |

Assays may be obtained commercially from DiaSorin, Inc. (Stillwater, Minn.), LabCorp (Dublin, Ohio) or other commercial laboratories, or they may be prepared by standard methods.

### ABBREVIATIONS

EBV, Epstein Barr virus
HCMV, Human cytomegalovirus
VCA, viral capsid antigen

### EA(D), Early Antigen Diffuse

### HHV6, Human Herpsevirus 6

Once it is determined which pathogens are present in a patient, appropriate pharmaceutical agents can be prescribed to treat each pathogen as described herein, according to embodiments of the present invention.

### Example 6

### CFS Multiplex RT-PCR Assay for Detection of HCMV, EBV and HHV6 Viral Transcripts

This assay can be used to detect HCMV, EBV and HHV6 viral expressed genes from a patient's circulating white blood cells, which can indicate active abortive infection caused by these viruses. It can be used as a confirmatory assay for infection with one or more of HCMV, HHV6, and EBV. Routine serum HCMV IgG and EBV VCA IgM (Viral Capsid IgM) or EBV EA(D) (Early Antigen (Diffuse)) can be used to identify CFS patients to be tested by this method. International and CDC criteria can be used to identify CFS patients.

Nucleic Acid Extraction

RNA is extracted from aliquots (0.2 ml) of plasma or whole blood on MagNA Pure extraction system using RNA extraction kit (Roche Molecular).

RT-PCR

This is a two-step reverse transcriptase polymerase chain reaction (RT-PCR) assay. cDNA is synthesized using Moloney murine leukemia virus RT (Invitrogen) in a 20 μl reaction mixture containing 0.5 μM random hexamers, 0.5 μM deoxynucleoside triphosphates (dNTP's), 1×RT buffer, 0.01 M dithiothreitol, and 5 μl of isolated viral RNA for 60 min at 37° C.

TSPE

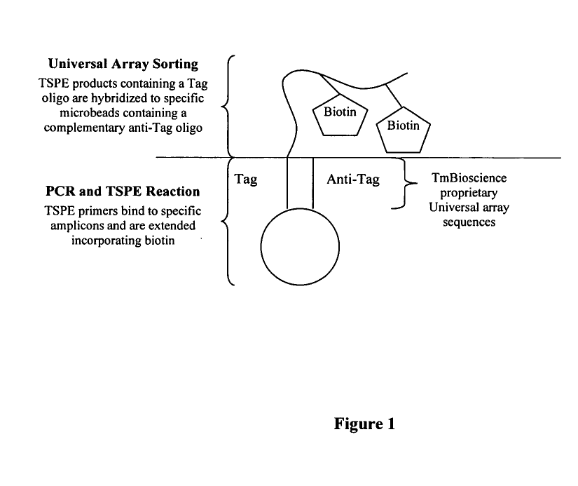

A multiplex target-specific primer extension (TSPE) reaction is used to detect specific viral sequences amplified by RT-PCR. Treated PCR products (5 μl) are added to a TSPE mixture that contains 12 oligonucleotide primers designed to recognize the 12 viral genes of HCMV, EBV and HHV6 set forth in Table 1; it also includes an internal control. (Other genes may be selected in addition to or instead of these.) TSPE primers are chimeric by design (Luminex Software for designing specific TSPE primers and probes), containing both a virus-specific oligonucleotide sequence and a tag oligonucleotide that hybridizes to a complementary anti-tag oligonucleotide bound to 12 spectrofluorometrically labeled microspheres (FIG. 1). The tag and anti-tag oligonucleotides can be, for example, proprietary sequences, for example those that are part of the Universal Array from TmBioscience Corporation (Toronto, Canada). These sequences are unique 3-base, 24-mer oligonucleotide pairs with isothermal hybridization kinetics. For the TSPE reaction, an aliquot (5 μl) of the treated PCR product is added to 15 μl of TSPE master mix, containing QIAGEN PCR buffer, dATP, biotinylated dCTP, dGTP, dTTP, 2.5 U TaKara hot-start polymerase, and TSPE primer mix (2.5 μM each). The reaction mixture is incubated with the following cycling conditions: 1 cycle for 2 min at 96° C. and then 35 cycles of 30 s at 54° C. and 30 s at 72° C. See FIG. 1.

Analysis of TSPE Products Using a Fluidic Microarray

Synthesized biotinylated TSPE products are hybridized to a fluid microbead-based array (part of the Universal Array) in wells of a 96-well microtiter plate and are detected using a strepto-vidin-phycoerythrin conjugate (Molecular Probes, Eugene, Oreg.). The CFS multiplex assay microbead mix consists of 12 microbeads, each containing a different fluorescent dye mix and each containing a unique anti-tag oligonucleotide sequence complementary to the oligonucleotide tag sequences incorporated into the 12 TSPE primers. A different number of microbeads is assigned to each primer pair. An aliquot of the TSPE reaction mixture (3.5 μl) is mixed with 20 μl of the microbead mix and incubated for 2 min at 96° C., followed by incubation for 30 min at 37° C. Aliquots (0.1 ml) of strepto-vidin-phycoerythrin conjugate diluted 1:100 in lμ wash buffer is added to the wells, and the plates are incubated in the dark for 20 min at room temperature. The washed plates are then read with a Luminex 100 flow cell instrument after 20 min of incubation. TSPE products bound to specific beads are detected by two lasers in the Luminex 100 flow cell instrument, the first being a red laser used to identify individual microbeads based on unique spectrophotometric dyes incorporated into the microbeads and the second being a green laser used to detect phycoerythrin fluorescence bound to each microbead. The signal on each bead is recorded as the mean fluorescence intensity (MFI) and the output, determined by TDAS software (TmBioscience Corporation), of the instrument that analyzes the raw data and makes a positive or negative determination for each virus gene.

Confirmatory PCR

There are twelve confirmatory RT-PCR assays, each using a unique pair of primers for each of the test viral genes to confirm positives detected by the CFS multiplex assay. Primers are designed the same way as those used in the CFS multiple assays, and optimal conditions for amplification are determined for each set of primers. For the two-step RT-PCR assays, cDNA is synthesized as described elsewhere for the CFS multiplex assay using random hexamers, and PCR is performed under optimal conditions (primer concentration and annealing temperature) derived for each set of primers. The analytical sensitivity for each confirmatory assay should be similar to the analytical sensitivity for the corresponding target in the CFS multiplex assay, as determined by end point titrations using serial dilutions of transcripts (Invitrogen kit) generated in vitro with cloned amplicons. Amplicons of targeted genes are cloned in the specific cloning vectors (plasmid). Cloned amplicons contain primer sequences that allow primers to bind and amplify the target. Therefore, these plasmids with the cloned amplicons can be used as an assay control.

Synthesized cDNA (5 μl) is amplified in a multiplex PCR using sequence specific primers designed to amplify highly conserved regions of viral genes for detection of HCMV, EBV and HHV6. The multiplex PCR can target, for example, the viral genes set forth in Table 1. The primers are chosen carefully for target specificity and size so that amplicons are small (100 to 400 bp) to maximize the amplification efficiency. In addition, primers specific for G6PDH are used to amplify human housekeeping gene G6PDH as an internal control that monitors the whole assay (extraction and amplification). G6PDH gene is cloned in the plasmid and is added to the extraction reaction. The PCR is run in 20 μl PCR master mix, consisting of 1.5 μl TaKara buffer, 0.15 mM dNTP's 1 U TaKara Taq polymerase, 12 pairs of primers at optimized concentrations ranging from 0.2 to 1 μM, and 5 μl of cDNA. The PCR cycling conditions are as follows: 1 cycle of 2 min at 95° C.; 30 cycles of 30 s at 95° C., 30 s at 55° C., and 30 s at 72° C.; and 1 cycle of 2 min at 72° C. Following PCR, the remaining dNTPs and primers are removed by incubating the entire 25-μl PCR mixture with 2.5 U shrimp alkaline phosphatase and 10 U exonuclease (Invitrogen) for 30 min at 37° C., followed by 30 s at 99° C.

### Example 7

### Cloning and Expression of Viral Antigens

The following methods produce the specific EBV, HCMV and HHV6 gene products (for example, the four exemplary gene products per herpesvirus listed in Table 1) to be used in ELISA tests of CFS serum.

DNA fragments coding for the different (early, middle and late) HCMV, EBV and/or HHV6 gene products selected as antigens are generated by PCR amplification using sequence specific primers as previously described. The general cloning strategy and the detailed methods have been described elsewhere. Briefly, amplification is performed with pairs of PCR primers containing recognition sequences of the restriction endonucleases BAMHI and EcoRI (Aval), respectively 5′ and 3′ of the original priming sequence, to facilitate the following cloning steps. Published viral sequences (NIH Genebank) with defined genomic fragments or cDNAs of selected viral genes serve as templates for PCR amplification. After initial cloning and identification of recombinant clones with the standard vector pUC8, DNA fragments are subcloned without modification intopGEX-3×, a vector which enables the expression of polypeptides in fusion with GST.

Western Blots

Western blots are performed with unpurified bacterial lysates according to the method of Baer et al. To ensure comparable amounts of viral antigens in each sample, the bacterial lysates are prediluted until the different recombinant proteins appear as faint bands of comparable intensities in a Coomassie-stained sodium dodecyl sulfate (SDS)-polyacrylamide gel. A lysate of a clone expressing GST alone is included as a control in each Western blot to confirm expression. After separation by SDS-15% polyacrylamide gel electrophoresis (PAGE), the proteins are transferred to polyvinylidine difluoride membranes (Millipore) under semi-dry conditions. Membranes are then blocked for 1 hour with 3% bovine serum albumin (Sigma) and incubated overnight with 1:100-diluted human sera. After membrane washing with wash buffer, bound immunoglobulins are visualized with horseradish peroxidase-conjugated polyclonal rabbit anti-human IgG or IgM (Dako) that develop color when reacted with diaminobenzidine.

Isolation and Purification of Expressed Viral Antigens

Selected expressed viral antigens (fusion proteins with GST) are purified using affinity column chromatography. Briefly, expressed antigens are purified after lysis of bacteria by glutathione-Sepharose affinity chromatography. Insoluble antigens (if any) are purified by differential washing and centrifugation steps using variable concentrations of detergents and chao-tropic reagents at different pH values. The final purity of the recombinant proteins should be higher than 95%. Purity is also confirmed by Western blotting and ELISA.

ELISA with Purified Recombinant Fusion Proteins

Polystyrene microdilution plates (96 well each) (Nunc) are coated with 100 ng of purified Viral antigen-GST fusion proteins or purified GST as a control solubilized in 100 R1 of 0.01 M carbonate buffer, pH 9.5. ELISA plates are incubated for 2 h with 1:50-diluted human sera in a water bath at 40° C. to prevent nonspecific binding. Coded wells are washed with PBS twice. Bound immunoglobins are detected by incubation with IgG- and IgM-specific conjugates (Dako) for 30 min at 40° C. Detection is performed by exposure to 1, 2-phenylenediamine for 15 min at room temperature, which causes color (yellow to brown). All other reagents are standard components used in commercially available ELISA kits (anti-EBV recombinant; Diasorin). All optical densities (OD) reported here are measured at 495 nm (reference, 620 nm) and are uncorrected values. Samples with OD values above 0.5 and a GST-specific reactivity below 0.25 are considered positive patient samples. However, optimization of each sample individually and all together is performed.

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All patents, published applications and published articles cited herein are incorporated by reference in their entirety.

1-51. (canceled)

52. A method of diagnosing a patient with CFS, comprising the steps of:
determining the presence in the patient of primary abortive infection by one or more types of herpesvirus; and
determining the presence in the patient of co-infection by one or more secondary non-viral infectious agents.

53. The method of claim 52, wherein the herpesvirus is selected from the group consisting of EBV, HCMV, HHV6, and combinations thereof.

54. The method of claim 52, wherein the secondary infectious agent is selected from the group consisting of Borrelia burgdorferi, Streptococcus pyogenes, Babesia microti, Mycoplasma pneumoniae, Ehrlichia chaffeensis and combinations thereof.

55. The method of claim 52, wherein determining the presence of co-infection by one or more secondary infectious agents comprises at least one of (i) detecting the presence of an antibody that specifically binds to one or more of the secondary infectious agents, or (ii) assaying for nucleic acid molecules of one or more of the secondary infectious agents.

56. The method of claim 52, wherein determining the presence of primary infection comprises at least one of (i) assaying for nucleic acid molecules that indicate the abortive replication of one or more CFS-causing herpesviruses, or (ii) assaying for expression of one or more middle (E or L) herpesvirus genes in a tissue of the patient.

57. The method of claim 56, wherein assaying for expression of one or more middle (E or L) herpesvirus genes comprises at least one of (i) detecting mRNA of the gene or conducting an immunoassay that detects antibodies to a protein product of the gene.

58. The method of claim 56, wherein assaying for nucleic acid molecules is conducted at a time when immunological evidence of the CFS-causing agents cannot be detected.

59. The method of claim 52, comprising:
determining if the patient meets the criteria for CFS as established by the International Chronic Fatigue Syndrome Study Group; and
determining if the patient has not exhibited any significant improvement in the previous six months.

60. The method of claim 52, further comprising repeating the method after at least about 2 weeks.

61. A method of treating a CFS patient diagnosed according to claim 52, comprising:
administering to the patient a therapeutically effective amount of at least one antiviral agent, such that each primary infection found in the patient is effectively treated by at least one antiviral agent administered to the patient, and
administering to the patient a therapeutically effective amount of at least one pharmaceutical composition, such that each co-infection found in the patient is effectively treated by at least one pharmaceutical composition administered to the patient,
for a period of time effective to treat the CFS.

62. The method of claim 61, wherein the antiviral agent is selected from the group consisting of valacyclovir, valganciclovir, maribavir, foscarnet, famciclovir, and combinations thereof.

63. The method of claim 61, wherein the at least one pharmaceutical composition comprises one or more active agents selected from the group consisting of ceftriaxone, amoxicillin, penicillin G, ataquavone, doxycycline, azithromycin and combinations thereof.

64. The method of claim 61, further comprising monitoring the patient over a time course of treatment, wherein monitoring the patient comprises at least one of (i) observing the patient for side effects resulting from the administration of the at least one antiviral agent; (ii) determining the level of primary abortive infection by one or more types of herpesvirus after treatment; (iii) conducting an immunoassay on a patient sample to determine the presence or absence of antibodies to one or more gene products of CFS-causing herpesvirus and/or non-viral infectious agents; or (iv) assaying for a decrease in the patient's levels of one or more gene products of CFS-causing herpesvirus and/or non-viral infectious agents, and adjusting the dosage level of the at least one antiviral agent and/or the at least one pharmaceutical agent accordingly.

65. The method of claim 61, further comprising repeating the method at least every 6 months until the patient lacks symptoms of CFS.

66. A method of determining a therapeutic regimen for chronic fatigue syndrome in a patient, comprising testing a patient sample for the presence of nucleic acid molecules of one or more CFS-causing herpesviruses and one or more non-viral infectious agents, and categorizing the patient's CFS as (a) caused by one or more herpesvirus and no non-viral infectious agent; (b) caused by one or more herpesviruses and at least one non-viral infectious agent; (c) not caused by a herpesvirus and caused by at least one non-viral infectious agent; and (d) not caused by a herpesvirus and not caused by at least one non-viral infectious agent.

67. The method of claim 66, wherein the nucleic acid molecules comprise gene products from early or middle genes that are expressed during abortive herpesvirus infection.

68. The method of claim 66, wherein the CFS-causing herpesvirus is selected from the group consisting of HCMV, EBV, HHV6, and combinations thereof.

69. The method of claim 66, further comprising at least one of (i) confirming the presence of nucleic acid sequences using confirmatory RT-PCR, or (ii) analyzing the patient sample for the presence of antibodies that bind to a gene product of the nucleic acid molecules.

70. The method of claim 66, further comprising administering to the patient one or more of (i) at least one of an antiviral agent such that each herpesvirus found in the patient is effectively treated by at least one antiviral agent administered to the patient, and (ii) at least one pharmaceutical composition such that each non-herpesvirus infectious agent found in the patient is effectively treated by at least one pharmaceutical composition administered to the patient.

71. The method of claim 70, further comprising monitoring the patient over a time course of treatment, wherein monitoring the patient comprises at least one of (i) observing the patient for side effects resulting from the administration of the at least one antiviral agent; (ii) determining the level of primary abortive infection by one or more types of herpesvirus after treatment; (iii) conducting an immunoassay on a patient sample to determine the presence or absence of antibodies to one or more gene products of CFS-causing herpesvirus and/or non-viral infectious agents; or (iv) assaying for a decrease in the patient's levels of one or more gene products of CFS-causing herpesvirus and/or non-viral infectious agents, and adjusting the dosage level of the at least one antiviral agent and/or the at least one pharmaceutical agent accordingly.

72. A kit comprising:
means for detecting serologic evidence of HCMV;
means for detecting serologic evidence of EBV;
means for detecting serologic evidence of HHV6; and
means for detecting serologic evidence of at least one non-viral pathogen selected from the group consisting of Borrelia burgdorferi, Streptococcus pyogenes, Ehrlichia chaffeensis, Babesia microti, and Mycoplasma pneumoniae.
